(19) United States
(12) Patent Application Publication
(10) Pub. No.: US 2024/0277212 A1
(43) Pub. Date: Aug. 22, 2024

(54) INFRARED THERMAL ENDOSCOPY

(71) Applicant: OWL PEAK TECHNOLOGIES, INC., McLean, VA (US)

(72) Inventors: Timothy Harvey, McLean, VA (US); Will Stewart, McLean, VA (US)

(73) Assignee: OWL PEAK TECHNOLOGIES, INC., McLean, VA (US)

(21) Appl. No.: 18/649,588

(22) Filed: Apr. 29, 2024

Related U.S. Application Data
(63) Continuation of application No. PCT/US2022/048286, filed on Oct. 28, 2022.
(60) Provisional application No. 63/273,821, filed on Oct. 29, 2021.

(51) Int. Cl. A61B 1/00; A61B 1/05; A61B 1/06

(52) U.S. Cl. CPC A61B 1/00097; A61B 1/000094; A61B 1/0014; A61B 1/05; A61B 1/0676

(57) ABSTRACT

The use of infrared (e.g., far-infrared) and temperature detection of abnormalities in an organ are disclosed herein. Systems, devices, and methods are provided leveraging the output of far-infrared detectors, and particularly, a plurality of far-infrared detectors to enhance abnormality detection in endoscopic procedures.

### CROSS REFERENCE TO RELATED APPLICATIONS

This application is a U.S. utility application under 35 U.S.C. 111(a) that is a Continuation of PCT International Patent Application No. PCT/US2022/048286, filed Oct. 28, 2022, designating the United States, and published in English, which claims priority to and the benefit of U.S. Provisional Patent Application No. 63/273,821, filed Oct. 29, 2021, the entire contents of which are hereby incorporated by reference in their entirety.

### FIELD OF DISCLOSURE

The present disclosure is related to infrared detection and, in particular, to medical devices and methods that utilize infrared imaging to measure thermally produced infrared radiation of abnormalities during endoscopic procedures.

### BACKGROUND

The term endoscopy broadly means “the visualization of a hollow organ or space with a scope.” For example, a colonoscopy typically enables an examination the lining of the rectum, all or part of the colon (large intestine) and even the lowest part of the small intestine (known as the ileum) for the detection of any abnormalities. During colonoscopy, an endoscope is inserted from the anus and then slowly advanced into the rectum, colon and in some cases the ileum. Typically, endoscopes comprise a flexible tube for insertion (a.k.a. “the insertion tube”) and a camera at its distal end, with a light source, configured to capture visual light images of the GI tract as the tube is inserted and removed therein. The insertion tube may be either rigid, flexible, or a combination of both, and the distal end of the insertion tube may additionally be steerable by the user. Most endoscopes have channels running along or inside the insertion tube to allow for the instillation of air, fluids, or even the insertion of instruments to assist in visualization or manipulation of tissue. There are specially-adapted endoscopes that are shaped and sized to facilitate the visualization of many organs and spaces in the human body, including but not limited to the small intestine, the brain, the sinus, the bronchus, the upper gut, the peritoneal cavity and even joint spaces.

However, endoscopes often have a limited field of view (FOV) typically oriented in the forward direction of the tube. In addition, due to various difficult conditions in the GI tract, such as sharp bends, folds, twists and turns in the colon, and ever the presence of fecal matter, it is difficult to maneuver the insertion tube for imaging the GI tract with complete coverage.

There is a special class of devices for endoscopy that do not have insertion tubes, but are instead designed to be swallowed or placed in a desired area difficult to reach with an insertion tube. These devices largely serve the same function of abnormality detection. These devices are commonly referred to as “capsule endoscopes” and their use is called “capsule endoscopy.”

Visible light and endoscope-based visual inspection of tissue and organs has its limitations. As described above, many areas of the body may be difficult to access with common endoscopes. This difficulty may arise due to the nature of the anatomy or the distances involved in traversing long hollow organs such as the small bowel. In addition, direct visualization using an endoscope may be limited by obstruction of visible light between the organ lining and endoscope camera, limitations of the endoscope camera and related technology, and also by subjectivity associated with an endoscopists interpretation of the images collected during the procedure. In particular, operators of endoscopes may miss regions of the organ or space containing abnormalities due to lack of image resolution, the presence of tissue folds, obstructions on the tissue surface (e.g., by deposition of fecal matter on the tissue surface that is not rinsed away), or fast distal tip movement speed.

Some endoscopic procedures have been developed to detect abnormalities using radiation other than visible light. For example, U.S. Pat. Nos. 8,774,902 and 10,791,916, which are hereby incorporated by reference in their entirety, detail the use of infrared sensors on endoscopes which detect the infrared radiation passively emitted from abnormalities having a temperature differential as compared to the surrounding tissue. However, these detection methods are difficult to implement in actual endoscopic procedures, often not providing enough information about an abnormality to identify abnormalities with non-visible light alone. The concept of “angiogenesis” broadly associates increased heat emission (in the form of infrared radiation) with abnormalities such as tumors, and the presence of increased IR in an area may provide invaluable information to clinicians who seek to assess abnormalities at a stage where visible light may be insufficient. Current and ongoing research points to the potential of IR to help identify cancer before it might be identified using traditional means. It has been found that many colon cancers discovered between the common 5 year intervals of standard endoscopy were due to lesions or polyps that were missed during that endoscopy. Nevertheless, implementation of IR measurements with endoscopic procedures has proven difficult and not gained large scale adoption. This may be due to a variety of reasons. For example, complexities associated with appropriately measuring IR throughout an organ or space, whether a set of IR parameters can accurately identify abnormalities during an endoscopy, and coupling IR information with the endoscopic images have made the use of infrared analysis difficult during endoscopic procedures.

It is an object of this disclosure to describe imaging and analysis technologies for endoscopes and other visualization devices. The disclosure includes devices, software and systems for augmenting endoscopes to provide increased data acquisition and enhanced interpretation of that acquired data as compared to previously available endoscopic procedures. These technologies may accompany the visual light image data of an endoscope to afford more accurate detection of abnormalities in living tissue during use.

### SUMMARY

In accordance with the foregoing objectives and others, the present disclosure describes endoscopes or devices for attachment to endoscopes comprising a set of sensors configured for detection of heat. These elements may then be coupled with additional hardware and software to capture, store, analyze, and/or interpret the data. The analysis is typically presented to the user to assist in diagnostic or therapeutic decision-making, sometimes in conjunction with visible images taken from endoscope. Often abnormalities have an increased heat signature as compared to normal tissue. The devices of the present disclosure utilize this signature in order to add an additional parameter for detection of abnormalities. Without wishing to be bound by theory, collection of heat data from a plurality of sensors on the device as it moves through hollow organ or space during a procedure provides unique abnormality signatures. The present disclosure includes methods and systems for detecting and interpreting this heat data, optionally with optical images, to afford more accurate detection and a more complete picture of abnormalities during an endoscopy. By leveraging algorithms provided from the machine learning processes described herein, the devices of the present disclosure are able to collect and leverage the unique signatures associated with multiple sensor measurement resulting in simple and quicker abnormality detection as compared to devices without multiple sensors and/or the machine learning abnormality detection described herein.

Provided herein, is a tip section of the tubular portion of an endoscope, wherein the tip section comprises the distal end of the endoscope tube and the wall of the tube proximal thereto; said tip section comprising:
a) a plurality of far-infrared sensors and/or temperature sensors distributed around said wall;
b) optionally a camera positioned on the distal end for imaging objects in front of the tube (e.g., the field of view of the camera includes the major longitudinal axis of the tube that extends past the distal end);
   wherein the plurality of far-infrared sensors, temperature sensors (e.g., one or more temperature sensors), and camera are configured each to transmit data for processing and analysis in conjunction with one another. The endoscope may include a visual light source for reflectance of the organ or space being imaged and into the camera for the creation of optical images. . . . In various implementations, the field of view of at least one infrared sensor includes an axis perpendicular to the wall surface or perpendicular to the longitudinal axis of the tube. In some embodiments, the field of view of each of the plurality of far-infrared sensors and the camera do not overlap. In other embodiments, the field of view of at least one of the sensors of plurality of far-infrared sensors and the camera overlap. The far-infrared sensors (or a portion thereof) may be disposed linearly (e.g., along the major longitudinal axis of the tube), circumferentially, partially circumferentially around the tube wall, or a combination thereof. In some embodiments, the far-infrared sensors (or a portion thereof) may be attached to the distal end of the endoscope. In various implementations, at least one far-infrared sensor may have a field of view that overlaps with the field of view of the camera. In some embodiments, the far-infrared sensors (or a portion thereof) may be disposed circumferentially around the tube wall such that each sensor lies in a plane perpendicular to the major longitudinal axis of the tube. In various implementations, the far-infrared sensors (or a portion thereof) may be disposed circumferentially around the tube wall such that each sensor lies in a plane that is not perpendicular to the major longitudinal axis of the tube. In some embodiments, the plurality of far-infrared sensors are integrated into the tubular and/or steering portion of the endoscope, such that the infrared sensors do not increase the diameter of the tubular portion. In some embodiments, the distal tip comprises from 1 to 15 far-IR sensors (e.g., from 2 to 15 far-IR sensors, from 2-10 far-IR sensors, 8 far-IR sensors).

Devices for attachment to the tubular portion of an endoscope are also provided. These devices are attachable (e.g., removably attached) to a wall proximal to the distal end of an endoscope tube. Typically, the endoscope may comprise a camera positioned on the distal end for imaging objects in front of the tube (e.g., the field of view of the camera includes the major longitudinal axis of the tube that extends past the distal end); and the device for tip section augmentation comprises a plurality of far-infrared sensors distributed on a substrate; and said substrate is attachable to the wall proximal to the distal end of the endoscope; wherein the plurality of far-infrared sensors, temperature sensors (e.g., one or more temperature sensors), and camera are configured each to transmit data for processing and analysis in conjunction with one another. In various implementations, the field of view of at least one infrared sensor includes an axis perpendicular to the wall surface and/or perpendicular to the longitudinal axis of the tube. In some embodiments, the field of view of each of the plurality of far-infrared sensors and the camera do not overlap. In other embodiments, the field of view of at least one of the sensors of plurality of far-infrared sensors and the camera overlap. The far-infrared sensors (or a portion thereof) may be disposed circumferentially or partially circumferentially around the tube wall. In some embodiments, the plurality of far-infrared sensors are integrated into the tubular portion of the endoscope, such that the device does not increase the diameter of the tubular portion or increases the diameter or circumference of the tube by less than (or from 0.1% to) 20% or less than 10% or less than 5%. In some embodiments, the far-infrared sensors (or a portion thereof) may be disposed circumferentially around the tube wall such that each sensor lies in a plane perpendicular to the major longitudinal axis of the tube. In various implementations, the far-infrared sensors (or a portion thereof) may be disposed circumferentially around the tube wall such that each sensor lies in a plane that is not perpendicular to the major longitudinal axis of the tube. In various implementations, the plurality of far-infrared sensors and/or thermal sensors are disposed on an ingestible camera such as a pill shaped camera configured to be ingested, pass through the GI tract and record far-infrared and thermal measurements of the tract as the ingestible camera passes from mouth to anus. In other implementations, the sensors described may be disposed and arrayed to replace visible light cameras entirely. In some embodiments, the device comprises from 1 to 15 far-IR sensors (e.g., from 2 to 15 far-IR sensors, from 2-10 far-IR sensors, 8 far-IR sensors).

These devices may, for example, be attached to an endoscope having a camera, but the endoscope may be moved through the organ or space without the camera in use. The housing may comprise a cavity where the distal tip of an endoscope may be inserted therein. The housing may also comprise a hole on a surface adjacent to the cavity such that the endoscope camera and/or visible light source may be aligned when inserted to maintain camera functionality when the device is attached to the endoscope. In some embodiments, the device may comprise a light source (e.g., a visible light source, an infrared light source) where reflected light may be sensed by the camera and/or plurality of IR sensors.

The relative orientation and fields of view of the infrared (e.g., far-infrared) sensors between one another an these devices may be a variable used to gather and interpret information about any abnormality detected by the sensors. For example, the field of view of at least two of said plurality of far-infrared sensors overlap such that data of an object may be acquired by different sensors simultaneously. The sensor configuration and overlap of fields of view may be a parameter used in the detection algorithm. Additionally, in some embodiments, overlapping fields of view may allow for detection of device problems such as sensor malfunction or obstruction (e.g., obstruction caused by deposition of in vivo material such as fecal matter on a sensor or along the tissue surface). In some embodiments, the identification of an anomalous temperature (such as an anomalous temperature viewed through an obstruction deposited on the tissue surface) may provide a notification to an endoscope operator to, for example, clear an area of potential interest from obstruction (e.g., via rinsing) and/or reorient the visual portion of the endoscope at a specific location and subsequent visual inspection of the area one or more times (e.g., following the obstruction clearing). In some embodiments, the distal tip is automatically repositioned for rinsing and visual inspection upon identification of an anomalous temperature. Additionally, for obstructions on a sensor that are difficult to remove during an endoscopic procedure, the machine learning algorithms may be able to identify when such obstruction occurs and/or compensate for detection using the remaining unobstructed sensors. These may, for example, recalculate the total field of view from the plurality of unobstructed IR sensors during the endoscopic procedure, or provide feedback that would notify the user to rinse or otherwise clean the sensor area. In some embodiments, the field of view of at least two of said plurality of far-infrared sensors may not overlap, but capture heat or temperature information from adjacent portions of an organ. In various implementations, at least two far-infrared sensors such that a first sensor images a observed portion of the organ, then, following motion of the endoscope (e.g., rotation, forward motion, reverse motion), the field of view of the second sensor may image the same or substantially the same observed portion of the organ. In some embodiments, a portion (e.g., a first portion, a second portion) of said plurality of sensors are disposed linearly along the wall such that said linear dispersion is substantially parallel (e.g., ±5°, ±1°) with the major longitudinal axis of the tube when said device is attached to the endoscope.

In some embodiments, at least two of the plurality of sensors detect different wavelengths of IR light. For example, each sensor may independently detect far IR (e.g., light having a wavelength of from 15-1000 μM), mid IR (e.g., light having a wavelength range of from 1,000 nm to 15,000 nm), or near IR (e.g., light having a wavelength range of from 800 nm to 3,000 nm). In some embodiments, more than 50% (e.g., from 50%to 100%, from 60% to 100%, from 70% to 100%, from 80% to 100%, from 90% to 100%) of the sensors on the device may detect far IR light (e.g., light having a wavelength of from 15-1000 μM or from 15-100 μm, from 100-200 μm, from 200-300 μm, from 300-400 μm, from 400-500 μm, from 500-600 μm, from 600-700 μm, from 700-800 μm, from 900-1000 μm). In some embodiments, sensors having overlapping fields of view detect the same wavelength range, different wavelength ranges, or combinations thereof.

The endoscope tube may be a tube having a curved outermost wall such as a cylindrical tube. In some embodiments, the cylindrical tube is an elliptic cylinder or a circular cylinder. For devices designed to attach to these tubes, the substrate may be dimensioned to match the geometry such as in a circular or elliptical ring designed to fit at a wall position of the tube proximal to the distal end.

The present disclosure is partially based on the discovery that specific far-infrared sensors provide better evaluation of abnormal heat signatures at physiologically relevant distances. For example, at least one of the far-infrared sensors may have a field of view less than (or from 0.1° to) 25° (e.g., less than 22°, less than 18°, less than 15°, from 1° to 20°, from 4° to 13°, from 5° to 12°). In various implementations, at least 50% (e.g., at least 60%, at least 70%, at least 80%, at least 90%, all) of said plurality of far-infrared sensors independently have a field of view less than (or from 0.1° to) 25° (e.g., less than 22°, less than 18°, less than 15°, from 3° to 22°, from 1° to 20°, from 4° to 13°, from 5° to 12°, from 1° to 2°, from 2° to 3°, from 3° to 4°, from 4° to 5°, from 5° to 6°, from 6° to 7°, from 7° to 8°, from 8° to 9°, from 9° to 10°, from 10° to 11°, from 11° to 12°, from 12° to 13°, from 13° to 14°, from 14° to 15°, from 15° to 16°, from 16° to 17°, from 17° to 18°, from 18° to 19°, from 19° to 20°, from 20° to 21°, from 21° to 22°, from 22° to 23°, from 23° to 24°). In certain embodiments, at least 90% (e.g., all) of said plurality of far-infrared sensors independently have a field of view from 5° to 12°. In various implementations, at least one, a portion of (e.g., more than 50%, more than 60%, more than 70%, more than 80%more than 90%), or all of the plurality of infrared sensors have a detection rate of at least 1 Hz (e.g., at least 2 Hz, at least 3 Hz, at least 4 Hz, at least 5 Hz, at least 6 Hz, at least 7 Hz, at least 8 Hz, at least 9 Hz, at least 10 Hz, from 2 Hz to 20 Hz, from 3 Hz to 20 Hz, from 4 Hz to 20 Hz, from 5 Hz to 20 Hz, from 6 Hz to 20 Hz, from 7 Hz to 20 Hz, from 8 Hz to 20 Hz, from 9 Hz to 20 Hz, from 10 Hz to 20 Hz).

The analysis for the additional heat parameter may be performed by endoscope systems. These endoscope systems may comprise:
a) an endoscope having the tip section of the present disclosure or a device of the present disclosure attached thereto;
b) a machine-readable medium configured to receive the data transmitted from the plurality of far-infrared sensors and, optionally, a camera; and
c) a processor comprising instructions to analyze the data transmitted to the machine readable medium to identify abnormalities of an organ or series of organ (e.g., the GI tract of a subject) based on the data transmitted from the plurality of far-infrared sensors and, optionally, the camera provided (e.g., conditions that exist on the tissue surface of the organ such as polyps, conditions that exist below the tissue surface, tumors, cysts, granulomas, abnormal circulatory vessels, inflammation).
   In some embodiments, the processor may comprise instructions for a camera AI algorithm to detect abnormalities from the camera images transmitted to the machine readable medium. For example, the processor may comprise instructions for a far-infrared AI algorithm to detect abnormalities data transmitted from the plurality of sensors to the machine readable medium. In various implementations, the processor comprises instructions for comparing the output of the camera AI algorithm and the far-infrared AI algorithm. In some embodiments, the instructions to analyze the data and identify abnormalities includes a calculation involving the position of the distal tip in the hollow organ or space (e.g., GI tract) and/or movement speed (e.g., forward speed, reverse speed, rotation speed) of the distal tip during data collection.

Methods of using an endoscope system to view an object are also provided comprising:
a) positioning an endoscope having a plurality of far-infrared sensors distributed around or in a wall proximal to the distal end of the endoscope tube; wherein the endoscope is positioned such that the object is in the field of view of at least one of the plurality of far-infrared sensors to detect temperature data of the object; and
b) transmitting the heat data to a machine readable medium.

The method of using an endoscope system to view an object may comprise:
a) providing an endoscope having a tip section of the present disclosure or a device of the present disclosure attached thereto having a plurality of sensors which detect heat or temperature data;
b) positioning a light source capable of emitting light (e.g., white light, red light, blue light, green light, infrared light, near-infrared light), wherein light emitted from the light source is reflected off the object and into the camera and/or plurality of far-IR sensors (or a portion thereof) to form image data and/or collect reflection data at far-IR wavelengths;
c) transmitting the image data to a machine readable medium;
d) optionally moving the distal tip of the endoscope such that the object is in the field of view of one or more of the far-infrared sensors to detect heat data of the object; and
e) transmitting the heat or temperature data to a machine readable medium.

In some embodiments, the method of using an endoscope system to view an object may comprise:
a) positioning an endoscope having a tip section of the present disclosure or a device of the present disclosure attached thereto, wherein the endoscope is positioned such that the object is in the field of view of at least one of the plurality of far-infrared sensors to detect heat data of the object;
b) transmitting the heat data to a machine readable medium;
c) positioning a light source capable of emitting light (e.g., white light, red light, blue light, green light, infrared light, near-infrared light), wherein light emitted from the light source is reflected off the object and into the camera to form image data;
d) transmitting the image data to a machine readable medium;
   In various implementations, the light source is positioned by movement of the distal tip. The light source may be positioned prior to measurement and analysis of the heat data and/or prior to measurement and analysis of the heat data. In some embodiments, the distal tip may be moved such that the object passes through the field of view of at least two far-infrared sensors in the plurality of far-infrared sensors. The positioning of the sensors may be effectuated before, during, or after identification of abnormalities with the camera. In some embodiments, analysis is performed on data taken by both the camera and the plurality of IR sensors (e.g., far-IR sensors) and movement is independent thereof. In some embodiments, the method may involve an alteration of the tissue surface such as the beginning of a rinsing step (e.g., a liquid such as saline is ejected from the distal tip to remove deposited matter from a tissue surface and/or a sensor) following identification of an abnormality via from the far-IR sensor data.

Typically, the methods involve collection of heat data from each sensor during said movement and transmitted to said machine readable medium. In some embodiments, the machine-readable medium is in communication with a processor comprising instructions to analyze the heat data and/or image data. In some embodiments, the heat and/or temperature data identifies an abnormality (e.g., polyp, subcutaneous abnormality) in a location (e.g., location of tissue) and the light source is positioned to reflect off the abnormality identified and into the camera. In some embodiments, the positioning of the light source involves a positioning of both the light source and the camera (e.g., via movement of the distal tip).

The endoscopists may choose to rinse any location (e.g., with a saline solution exiting a nozzle proximal to the distal tip) of tissue to clear obstructions and/or gain a better view of the location being imaged. For example, in some embodiments, the method may further comprise rinsing the location. In some embodiments, the rinsing may occur before positioning the light source to view the abnormality location. In other embodiments, the rinsing may occur after positioning the light source to view the abnormality location, and the method further comprises re-positioning the light source to reflect off the abnormality identified and into the camera to view the rinsed abnormality location. Typically, these methods comprise transmitting and/or displaying the heat and/or temperature and/or image data to an interface (e.g., graphical user interface) for an endoscope user to view (e.g., in real-time) an object identified in the data (e.g., an abnormality, a rinsed abnormality, a potential abnormality). In some embodiments, the data is stored and can be viewed at a later time (e.g., for use in the machine learning algorithms described herein).

Embodiments of the invention generally include infrared imaging devices that are configured for side-scan infrared imaging for, e.g., medical applications such as those described in U.S. Pat. No. 10,791,916, which hereby incorporated by reference in its entirety. For example, in one embodiment of the invention, an imaging device includes a ring-shaped detector element comprising a circular array of infrared detectors configured to detect thermal infrared radiation, and a focusing element configured to focus incident infrared radiation towards the circular array of infrared detectors.

### BRIEF DESCRIPTION OF FIGURES

FIGS. 1A-E provide cross sectional views of tip sections of endoscopes comprising arrays of far-infrared detectors in certain geometries.

FIGS. 2A and B provide views of an endoscope comprising a device having a plurality of sensors attached to its distal tip. FIG. 2A provides a top view of the device attached to the endoscope, and FIG. 2B provides a front view of the device and distal tip.

FIGS. 3A and B provide views of an endoscope wherein the plurality of sensors are distributed around the housing of the distal tip.

FIG. 4 provides an exemplary flow chart of an endoscopic procedure using the devices, systems, and methods of the present disclosure.

FIG. 5 provides an exemplary flow chart of an exemplary machine learning procedure to construct the calculation units used for abnormality detection.

FIG. 6 shows the sensor data measured of various sensors as a function of distance from a heat source.

FIGS. 7A and 7B are schematic diagrams of the sensor measurement described in the Examples.

FIGS. 8A and 8B provide the thermal far-infrared IR measurements taken with a 5° FOV sensor as scanned across a heated mask comprising a hole for abnormality simulation at 0″ from the heated mask (FIG. 8A) and at 1″ from the heated mask (FIG. 8B).

FIGS. 9A and 9B provide the thermal far-infrared IR measurements taken with a 12° FOV sensor as scanned across a heated mask comprising a hole for abnormality simulation at 0″ from the heated mask (FIG. 9A) and at 1″ from the heated mask (FIG. 9B).

FIGS. 10A and 10B provides the thermal far-infrared IR measurements taken with various Melexis sensors as scanned across a heated mask comprising a hole for abnormality simulation at 0″ from the heated mask (FIG. 10A) and at 1″ from the heated mask (FIG. 10B).

FIGS. 11A and 11B provide the thermal far-infrared IR measurements taken with a 12° FOV Melexis sensor as scanned across a heated mask comprising a hole for abnormality simulation at 0″ from the heated mask (FIG. 11A) and at 1″ from the heated mask (FIG. 11B).

FIGS. 12A and 12B provide the thermal far-infrared IR measurements taken with a 5° FOV sensor as scanned across a heated mask comprising a hole for abnormality simulation at 0″ from the heated mask (FIG. 12A) and at 1″ from the heated mask (FIG. 12B).

FIG. 13A provides an image of a synthetic bowel with a resistive heater disposed at the back to simulate an abnormality. FIG. 13B provides the negative control of a scan of this protocol as scanned over the surface shown in FIG. 13A with all components at ambient temperature.

FIGS. 14A-D provide sensor measurements of 5° and 12° FOV Melexis sensors over the synthetic bowl during abnormality simulation.

FIG. 15A is an image of the curved experimental setup of the synthetic bowel material and FIG. 15B is a thermal image of the curved material with a resistive heat source applied on the opposite side of the material.

FIG. 16 provides far-infrared sensor scans of the curved synthetic bowel having a simulated abnormality.

FIGS. 17A and 17B demonstrate horizontal (FIG. 17A) and vertical (FIG. 17B) configurations of a plurality of sensors with respect to an abnormality.

FIGS. 18A and 18B provide sensor measurements of the three sensors as scanned over a curved synthetic bowel having a simulated anomaly in the horizontal (FIG. 18A) and vertical (FIG. 18B).

FIGS. 19A-D provide views of the sensor fixture (sensor housing and sensor array) used in the in vivo abnormality simulation experiments.

FIGS. 20A-D provide views of the sheath used in the in vivo abnormality simulation experiments.

FIGS. 21A-C provide views of the attachment designed to orient the sensor fixture with respect to the simulated abnormality within the intestine in the in vivo abnormality simulation experiments. FIGS. 21D-E are views illustrating how the interface between the sensor fixture, sheath, and attachment operate to allow for sensor/abnormality alignment in these experiments.

FIG. 22 provides an exemplary graphical user interface (GUI) used for the experiments enabling real time data monitoring and tagging of potential abnormalities based on the FAR-IR measurement data.

FIG. 23 shows the two different thermistor measurements during resistor heat calibration and equilibrium.

FIG. 24 provides an exemplary negative control experiment using a 5° FOV sensor.

FIG. 25 provides the measurement of the simulated abnormality during the in vivo experimentation. Vertical lines illustrate when the abnormality has entered the FOV of the sensor used.

FIG. 26 provides plots of data take from 10 different runs at location 3.

FIG. 27A provides two exemplary negative control runs using a 12° FOV sensor.

FIG. 27B provides the data taken of the simulated abnormality using a 12° FOV sensor. FIG. 27C provides the data from the 12° FOV sensor for ten different runs at location 6.

FIGS. 28A and B each provide the IR data sensor data and corresponding actual abnormality position (solid vertical lines), and estimated abnormality position based on a blind GUI user evaluating the real-time data (dashed horizontal lines). FIG. 28A shows the blinded measurement with a 5° FOV sensor and FIG. 28B shows the blinded measurement with a 12° FOV sensor.

FIGS. 29A and 29B compare the raw data taken for FAR-IR simulated abnormality measurements and a machine learning based algorithm which identifies abnormality (or “outlier”) position of this raw data. In FIG. 29B, the boxed data is the data identified by the machine-learning algorithm as an outlier.

FIG. 30A provides the data from ten fully blinded trials taken from experiments performed with the bowel placed in abdominal cavity. FIG. 30B provides an exemplary fully blinded trial run with the estimated polyp location identified by a GUI operator (vertical dashed lines).

FIG. 31 is a plot of the FAR-IR measurements following saline application to the interior cavity prior to measurement.

FIG. 32 is a plot of the FAR-IR measurements of two runs from the application of fecal matter to the sensor face.

FIGS. 33A-D provide plots of the thermal measurements of polyps as performed by thermal sensors used during colonoscopies. Boxed data is associated with polyps.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all % of individual components of a plurality will not exceed 100% unless indicated otherwise.

In this disclosure, “infrared radiation” (within the context of its measurement during an endoscopy), “IR” (within the context of its measurement during an endoscopy), “heat signature,” and “heat data” may be used interchangeably, unless otherwise indicated. However, “heat data” or “heat signature” may include additional parameters associated with the IR measurement such as relative changes of temperature in relation to ambient temperature, the fields of view of one or more sensors, movement of the plurality of IR sensors, or temperature measurements performed by other methods (e.g., by the temperature sensors).

The tip sections of the present disclosure may comprise the distal end of the endoscope tube and the wall of the tube proximal thereto; said tip section comprising:
a) a plurality of far-infrared sensors and/or one or more temperature sensors distributed around said wall; and
b) optionally a camera positioned on the distal end for imaging objects in front of the tube (e.g., the field of view of the camera includes the major longitudinal axis of the tube that extends past the distal end);
   wherein the plurality of infrared sensors (e.g., far-infrared sensors), temperature sensors, and camera are configured each to transmit data for processing and analysis in conjunction with one another. In various implementations, the field of view of at least one infrared sensor includes an axis perpendicular to the wall surface or perpendicular to the longitudinal axis of the tube. In some embodiments, the field of view of each of the plurality of far-infrared sensors and the camera do not overlap. In other embodiments, the field of view of at least one of the sensors of plurality of far-infrared sensors and the camera overlap. The far-infrared sensors (or a portion thereof) may be independently disposed circumferentially or partially circumferentially around and/or within the tube wall. In some embodiments, the far-infrared sensors (or a portion thereof) may be independently disposed circumferentially around and/or within the tube wall such that each sensor lies in a plane perpendicular to the major longitudinal axis of the tube. In various implementations, the far-infrared sensors (or a portion thereof) may be independently disposed circumferentially around and/or within the tube wall such that each sensor lies in a plane that is not perpendicular to the major longitudinal axis of the tube.

In some implementations, the tip section may comprise a ring-array type structure of far-infrared sensors. Such tip sections may be used in conjunction with an endoscope camera and/or ingestible camera and/or capsule endoscope. An endoscope, for example, may comprise a housing on the tube, fiber optics for transmission of light information to or from the distal end and/or wires for electrical communication to the distal end including the plurality of far-infrared detectors, lenses connected to the fiber optics. The plurality of far-infrared sensors and/or temperature sensors may be positioned around and/or within the tube housing and be configured for communication through the housing (e.g., via wires) and/or for wireless transmission of data. Electrical wiring and interconnections can extend through the housing to provide power to the thermal IR ring-array imaging device and transmit real time data of thermal IR captured by the plurality of far-infrared sensors optionally in conjunction with image data from an endoscope camera.

The devices for augmentation of an endoscope may be attachable (e.g., removably attached) to a wall proximal to the distal end of an endoscope tube. Typically, the endoscope may comprise a camera positioned on the distal end for imaging objects in front of the tube (e.g., the field of view of the camera includes the major longitudinal axis of the tube that extends past the distal end); and the device for tip section augmentation comprises a plurality of far-infrared sensors distributed on a substrate; and said substrate is attachable to the wall proximal to the distal end of the endoscope; and

wherein the plurality of far-infrared sensors, temperature sensors, and camera are configured each to transmit data for processing and analysis in conjunction with one another. In various implementations, the field of view of at least one infrared sensor includes an axis perpendicular to the wall surface or perpendicular to the longitudinal axis of the tube. In some embodiments, the field of view of each of the plurality of far-infrared sensors and the camera do not overlap. In other embodiments, the field of view of at least one of the sensors of plurality of far-infrared sensors and the camera overlap. The far-infrared sensors (or a portion thereof) may be disposed circumferentially or partially circumferentially around the tube wall. In some embodiments, the far-infrared sensors (or a portion thereof) may be disposed circumferentially around the tube wall such that each sensor lies in a plane perpendicular to the major longitudinal axis of the tube. In various implementations, the far-infrared sensors (or a portion thereof) may be disposed circumferentially around the tube wall such that each sensor lies in a plane that is not perpendicular to the major longitudinal axis of the tube.

The delivery assembly may operate in conjunction with endoscopes, such as for example currently available endoscopes. Another aspect of the current invention provides a delivery assembly that attaches easily to currently available endoscopes without generally requiring modification of such endoscopes. Another aspect of the current invention provides an endoscope delivery assembly that is easily used and requires minimal training of the endoscopist. Generally, these assemblies involve a substrate comprising a plurality of far-infrared sensors disposed therein or around, wherein the substrate is dimensioned and comprises elements for removable attachment to an endoscope (e.g., clips, screws).

In some implementations, the device may form a ring-array type structure which can be implemented with an endoscope device to provide real-time scanning. Such device may be used in conjunction with an endoscope camera. An endoscope may comprise a housing on the tube, fiber optics for transmission of light information to or from the distal end and/or wires for electrical communication to the distal end including the plurality of far-infrared detectors, lenses connected to the fiber optics. The ring device may fit around the housing and be configured for communication through the housing and/or for wireless transmission of data. The fiber optic bundle may pass through an opening in the substrate and pass through the interior of the ring-shaped detector element. The thermal IR ring-array imaging device is typically secured at the forward end of the endoscope. The detector circuitry (not shown) can be formed on the substrate. Electrical wiring and interconnections can extend through the housing to provide power to the thermal IR ring-array imaging device and transmit real time data of thermal IR captured by the plurality of far-infrared sensors optionally in conjunction with image data from an endoscope camera.

The infrared radiation (IR) band typically covers the wavelength range of 700 nm-1 mm, frequency range of 430 THz-300 GHz, and photon energy range of 1.7 eV-1.24 meV. Far-infrared radiation (FIR) is found on the wavelength spectrum at 1-1000 μm with a frequency range of 20-0.3 THz, and photon energy range of 83-1.2 meV. In some embodiments, each sensor in the plurality of near-infrared sensors detect and measure the presence of electromagnetic radiation having a wavelength of from 1 μm-1000 μm. In some embodiments, the endoscope may further comprise one or more infrared sensors including near-infrared sensors. In some embodiments, at least two of the plurality of sensors detect different wavelengths of IR light. For example, each sensor may independently detect far IR (e.g., light having a wavelength of from 15-1000 μM), mid IR (e.g., light having a wavelength range of from 1,000 nm to 15,000 nm), or near IR (e.g., light having a wavelength range of from 800 nm to 3,000 nm). In some embodiments, more than 50% (e.g., from 50% to 100%, from 60% to 100%, from 70% to 100%, from 80% to 100%, from 90% to 100%) of the sensors on the device may detect far IR light (e.g., light having a wavelength of from 15-1000 μM or from 15-100 μm, from 100-200 μm, from 200-300 μm, from 300-400 μm, from 400-500 μm, from 500-600 μm, from 600-700 μm, from 700-800 μm, from 900-1000 μm). In some embodiments, sensors having overlapping fields of view detect the same wavelength range, different wavelength ranges, or combinations thereof.

While the light camera can only be used to capture images of conditions that may exist on the tissue surface examined (e.g., the GI tract mucosa), the IR (e.g., far-infrared) sensors can be used to capture images of conditions that may exist below the tissue surface, for example, less than 4 mm (e.g., 0.5 mm to 4 mm) below the surface of the tissue. Indeed, imaging below the tissue surface can be performed by measuring emissive thermal electromagnetic radiation such as far-infrared. Typically, the far-infrared sensors may detect the thermal infrared portion of the electromagnetic heat spectrum having a wavelength of 2 μm to 15 μm. In addition, when viewing the spectrum that is emitted from living tissue, emissive photons and heat created by biological activity of living cells may be observed. This emissive IR heat can propagate through a certain thickness of tissue of the wall of the GI tract thereby providing a measurement of subsurface properties. The magnitude of the emissive IR heat will vary based on the density of the underlying cells and the type of biological activity that the tissue performs. By filtering the IR thermal view to specific wavelengths (e.g., 4 μm, 11 μm), the devices may isolate and view tissue of different density and heat emissivity, and thereby acquire useful data about potential abnormal conditions (e.g., tumor, cyst, granuloma, abnormal circulatory vessel, precancerous abnormalities, cancerous abnormalities, benign abnormalities, inflammation) that may exist in early stages below the surface of the tissue, and which may not be visibly exposed on interior surface at the time of measurement. The devices of the present disclosure may allow for early detection of these subsurface abnormalities before some later time when abnormalities grow and acquire more cellular activity. Advantageously, thermal IR imaging devices and techniques as described herein may allow for early detection of such abnormal conditions that exist below the surface tissue.

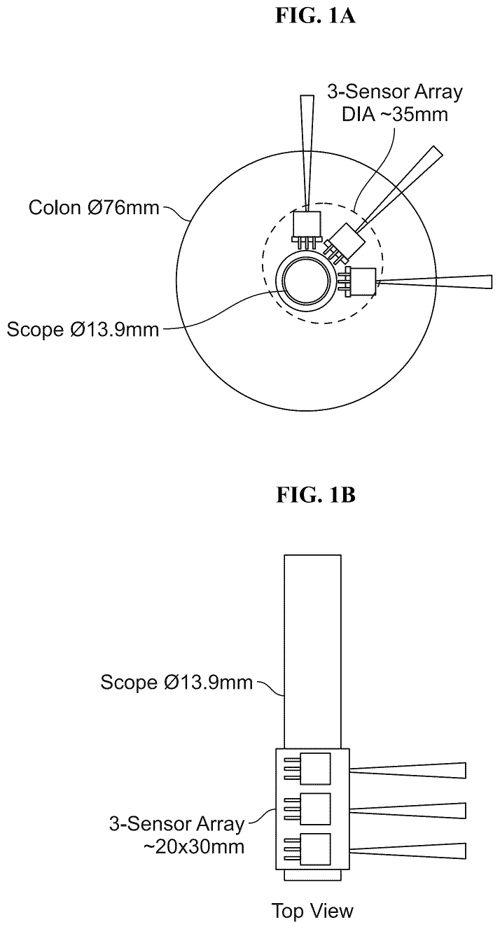

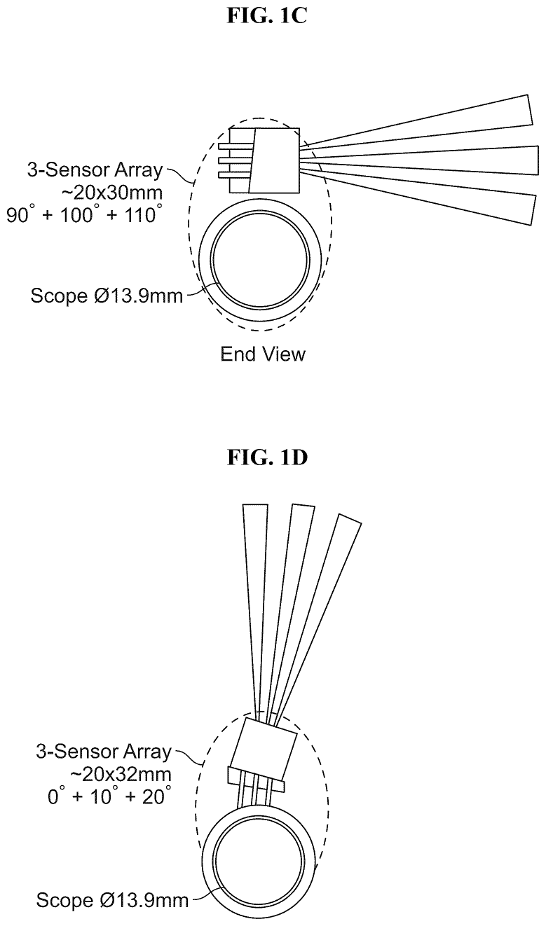

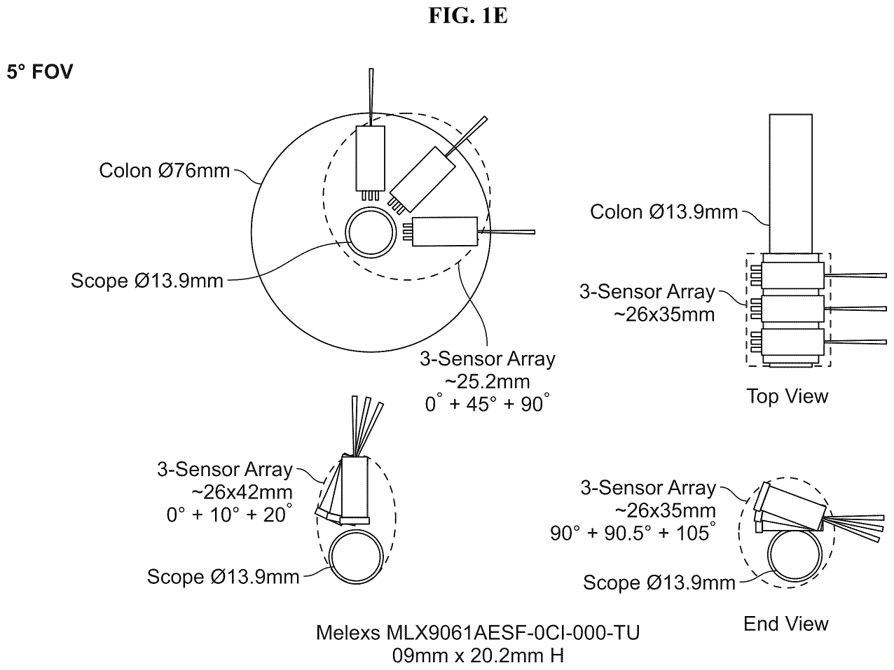

The plurality of sensors distributed on a tip section of the endoscope may comprise combinations of configurations described herein in order to increase the FOV of the endoscope. FIG. 1A provides the cross section of an exemplary three sensor array attached to an endoscope tube. In this embodiment, the endoscope tube has a 13.9 mm OD and the embodiment is illustrated sitting in a 76 mm diameter colon. With these three Melexis MLX90614ESF-DCH-000-TU 12° FOV sensors, the total tube has a diameter of 35 mm. The FOV for each individual sensor is shown illustrating the portions of the colon which may be measured when the array is positioned in this configuration. The sensors are oriented on the perimeter of the outer perimeter surface of endoscope tube in a plane perpendicular to the major longitudinal axis of the tube. These sensors are partially circumferential around the tube wall (i.e., they occupy only a portion of the circumference such as from 10% to 90% or from 20% to 80% of the circumference). These sensors may be housed in a container adapted to attach to the distal end of an endoscope. The container may be cylindrical, having a diameter that increases the diameter of the inserted device and endoscope by more than 1.1× (e.g., more than 1.5×, more than 2×, from 1.1× to 4×, from 1.5× to 3×) as compared to the distal end of the endoscope itself. For example, if a container were constructed to house the sensors such that they are fully embedded within the 35 mm diameter depicted, the increase in diameter is 2.5×(35 mm/13.9 mm=2.5×).

Different configurations of the sensors may be used in order to increase the total area of the organ measured by the plurality of sensors. For example, FIGS. 1B-D illustrate configurations where the sensors are oriented in a linear configuration along an axis parallel to the major longitudinal axis of the tube. FIG. 1B shows a top view of a suitable linear configuration, where each sensor's field of view is oriented tangentially or substantially tangentially (e.g., ±10° to the tangent of the tube defined at the central sensor position, ±5°, ±1°) to the tube wall. As the tube moves through a hollow organ, anomalies may enter each field of view allowing for multiple measurements to be integrated and aid in detection. When in this linear orientation, each FOV may be substantially parallel such that as the tube moves through the organ, the same region is monitored by each sensor. FIG. 1C shows an end view configuration of the sensors where the sensors are arranged in a linear arrangement, but each sensor has a rotated FOV with respect to the other sensors thereby providing measurements of adjacent positions of the organ. FIG. 1D shows an embodiment, where the FOV extends perpendicularly or substantially perpendicular to the endoscope tube tangent. In various implementations, the plurality of the far-infrared sensors (or a portion of the far-infrared sensors) are oriented circumferentially (or partially circumferentially) around the wall of the endoscope tube, linearly along the major longitudinal axis of the tube, or combinations thereof. In various implementations, the central axis of the FOV of each sensor independently parallel, substantially parallel, perpendicular, or substantially perpendicular with the tangent of the tube at the point of sensor attachment.

FIG. 1E provides similar schematics to FIGS. 1A-D for endoscopic tip sections of the present disclosure with dimensions illustrating Melexis MLX90614ESF-DCI-000-TU 5° FOV sensors. The plurality of far-infrared sensors may be chosen, for example from Excelitas 120° FOV sensors, Excelitas 5° FOV sensors, Melexis 50° FIV sensors, Melexis 35° FOV sensors, Melexis 90° FOV (DAA), Melexis 90° FOV sensors (DCA), Melexis 12° FOV sensors, and Melexis 5° FOV sensors. In particular embodiments, the infrared sensors are Melexis 12° FOV sensors and/or Melexis 5° FOV sensors. In certain implementations the sensors may be pixel sensor arrays such as the Excelitas 59×3° FOV sensor, the Excelitas 22° FOV sensors, or the Excelitas 17° FOV sensor. In various implementations, at least 50% (e.g., at least 60%, at least 70%, at least 80%, at least 90%, all) of said plurality of far-infrared sensors independently have a field of view less than (or from 0.1° to) 25° (e.g., less than 22°, less than 18°, less than 15°, from 3° to 22°, from 1° to 20°, from 4° to 13°, from 5° to 12°, from 1° to 2°, from 2° to 3°, from 3° to 4°, from 4° to 5°, from 5° to 6°, from 6° to 7°, from 7° to 8°, from 8° to 9°, from 9° to 10°, from 10° to 11°, from 11° to 12°, from 12° to 13°, from 13° to 14°, from 14° to 15°, from 15° to 16°, from 16° to 17°, from 17° to 18°, from 18° to 19°, from 19° to 20°, from 20° to 21°, from 21° to 22°, from 22° to 23°, from 23° to 24°). In certain embodiments, at least 90% (e.g., all) of said plurality of far-infrared sensors independently have a field of view from 5° to 12°. In various implementations, at least one, a portion of (e.g., more than 50%, more than 60%, more than 70%, more than 80%more than 90%), or all of the plurality of infrared sensors have a detection rate of at least 1 Hz (e.g., at least 2 Hz, at least 3 Hz, at least 4 Hz, at least 5 Hz, at least 6 Hz, at least 7 Hz, at least 8 Hz, at least 9 Hz, at least 10 Hz, from 2 Hz to 20 Hz, from 3 Hz to 20 Hz, from 4 Hz to 20 Hz, from 5 Hz to 20 Hz, from 6 Hz to 20 Hz, from 7 Hz to 20 Hz, from 8 Hz to 20 Hz, from 9 Hz to 20 Hz, from 10 Hz to 20 Hz).

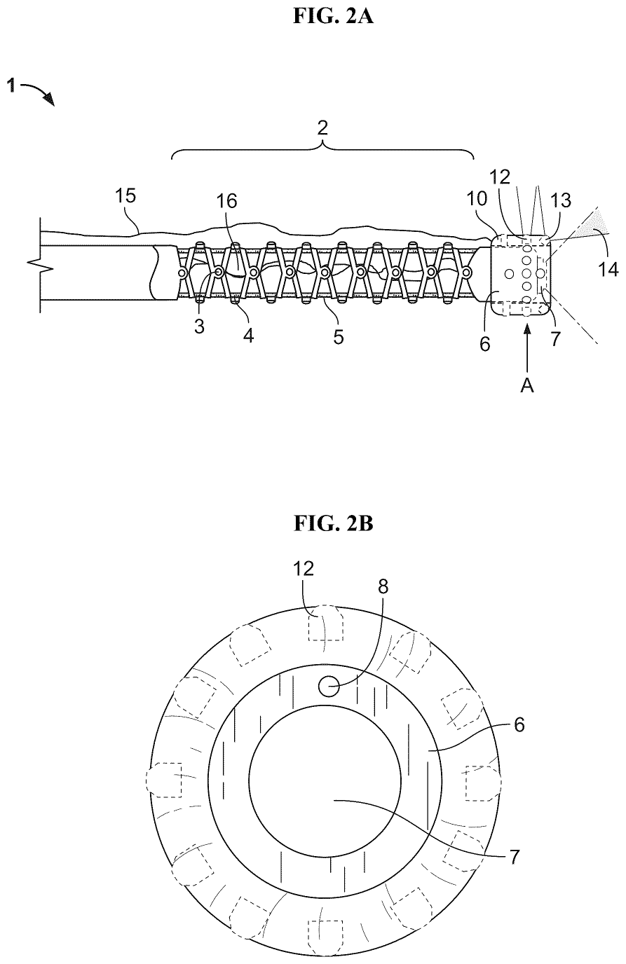

FIGS. 2A and B provide illustrations of a top view of a portion of an endoscope 1 (FIG. 1A) and a front view of the tubular portion of the distal tip and container along the major longitudinal axis of the endoscope. Endoscope 1 comprises bending section 2 and distal tip 5. Bending section 2 comprises pivot bins 3 and 4 and angulation wires 8 which allow for bending of the distal tip of the endoscope during use. Distal tip 6 houses camera 7 with a field of view indicated by the dashed dot lines and optical light source 8. Attached to distal tip 6 is device 10 comprising a plurality of far-infrared sensors including far-infrared sensors 12 and 13. Device 10 comprises a cavity dimensioned to accommodate distal tip 6 and hole connected to the cavity to allow for camera 7 and light 8 to still be operative. Device 10 provides receives power and transmits data through wires 15 and 16. Wire 15 is connected to the device and run exterior to the endoscope, while wires 16 are run internally through the endoscope. In some embodiments, the plurality of far-IR sensors receive power and transmit measurements via wires run internally through the device. In some embodiments, the device includes a battery to provide power to the far-IR sensors. In some embodiments, the far-IR sensors transmit data wirelessly (e.g., via bluetooth, wireless, RFID communication algorithms).

In FIG. 2A, a series of far-IR sensors positioned circumferentially around the distal end can be seen including far-IR sensor 12 and its corresponding Field of View (FOV). In FIG. 2B, the internal cross section positioning of the sensors containing axis A is shown illustrating a circumferential arrangement of 12 far-IR sensors like sensor 12 around the entire circumference of the distal tip. As can be seen, each of the far-IR sensors on device 10 are embedded within the device housing and have minimal (e.g., less than 5% change in diameter) effect on the external surface and configuration of the housing. In FIG. 2A three sensors can also be seen disposed linearly along the major axis of the device.

Far-IR sensor 12 has a central axis of a field of view oriented perpendicularly or nearly perpendicularly (e.g., from 80° to 100°) to the major longitudinal axis of distal tip 6. Far-IR sensor 13 has a central axis of a field of view that is not oriented perpendicularly (e.g., it forms an angle of from 10° to 80° or from 20° to 70°) with the major longitudinal axis of distal tip 6. The fields of view of each sensor may independently overlap or not overlap with any other sensor in the device. Generally, overlapping and nonoverlapping configurations of sensors such as adjacent sensors or sensors and camera may allow for a device or endoscope to take multiple measurements as necessary, which may independently be used to decrease false-positive rates of detection (e.g., by moving the tip to locations for different measurement determined, for example, by the machine learning algorithms trained on a specific geometry of far-IR sensors). As can be seen, the FOV of far-IR sensor 12 does not overlap with the FOV of far-IR sensor 13. However, the FOV of far-IR sensor 13 does overlap with the FOV of camera 7 in section 14. In particular embodiments, all far-IR sensors with overlapping fields of view have the same detection frequency. In other embodiments, at least two far-IR sensors with overlapping fields of view have different detection frequencies. These various parameters may be utilized by the machine-learning algorithms described herein to identify abnormalities.

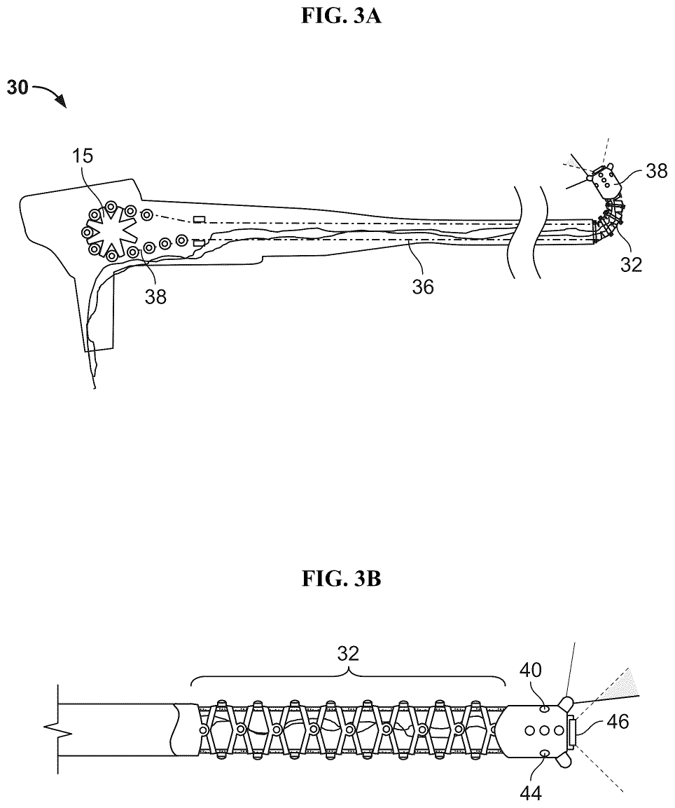

FIGS. 3A and B show an endoscope 30 having bending section 32 controlled by angulation knob 34 which moves angulation wire 36 via chain and sprocket. Movement of bending section 32 allows for the movement of distal tip 38 as the endoscope moves through the organ or space being examined. Distal tip 38 comprises a plurality of far-IR sensors including far-IR sensors 40, 42, and 44. Distal tip 38 also comprises camera 46. As can be seen, the FOV of sensor 42 and the field of view of camera 46 overlap. In the embodiment depicted, a portion of the plurality of far-IR sensors are embedded within the distal tip housing of the endoscope (e.g., far-IR sensors 40 and 44). Another portion of the plurality of far-IR sensors are positioned on the wall of the distal tip (e.g., far-IR sensor 42).

In both the endoscope and device for attachment to an endoscope embodiments of the present disclosure, each far-IR sensor may be independently embedded within or on a wall of the distal tip housing or device housing. In some embodiments, each far-IR sensor is embedded within the wall of the distal tip housing or device housing.

The analysis for the additional heat or temperature parameters measured may be performed by endoscope systems and methods of the present disclosure. These may comprise:
a) an endoscope having a tip section or a device of the present disclosure attached thereto;
b) a machine readable medium configured to receive the data transmitted from the plurality of far-infrared sensors and the camera; and
c) a processor comprising instructions to analyze the data transmitted to the machine readable medium to identify abnormalities of an organ or series of organ (e.g., the GI tract of a subject) based on data transmitted from the plurality of far-infrared sensors and, optionally, the image provided (e.g., conditions that exist on the tissue surface of the organ such as polyps, conditions that exist below the tissue surface, tumors, cysts, granulomas, abnormal circulatory vessels) by the camera.
   In some embodiments, the processor may comprise instructions for a camera AI algorithm to detect abnormalities from the camera images transmitted to the machine readable medium. For example, the processor may comprise instructions for a far-infrared AI algorithm to detect abnormalities data transmitted from the plurality of sensors to the machine readable medium. In various implementations, the processor comprises instructions for comparing the output of the camera AI algorithm and the far-infrared AI algorithm. In some embodiments, the instructions to analyze the data and identify abnormalities includes a calculation involving the position of the distal tip in the GI tract and/or movement speed (e.g., forward speed, reverse speed, rotation speed) of the distal tip during data collection.

The temperature sensors and/or far-IR sensors may be used to also collect thermal data regarding anomalies in the organ or space (e.g., GI tract). For example, the temperature sensor may be contacted with tissue (e.g., GI tract) (e.g., by the endoscope, by an ingestible camera) to register an anomalous temperature as compared to the surrounding tissue. For example, a difference as low as (or up to 10° C.) 0.1° C. or 0.5° C. or 1° C. (e.g., from 0.1° C. to 5° C., from 0.1° C. to 2° C., from 1° C. to 2° C., from 1° C. to 1.5° C.) may be used as a benchmark to identify an anomaly identified, for example, by movement of an endoscope tip or by travel of the ingestible pill through the GI tract. It will be understood that a temperature differential that can be converted into differences in Celsius will also be included within the scope of disclosure. For example, a difference as low as (or up to 10° C.) 0.1° C. or 0.5° C. or 1° C. (e.g., from 0.1° C. to 5° C., from 0.1° C. to 2° C., from 1° C. to 2° C., from 1° C. to 1.5° C.) can be measured and analyzed in relation to the raw output of one or more IR sensors (e.g., far-IR sensors) and/or temperature sensors (e.g., thermocouple). Upon comparison to surrounding tissue, as the temperature sensor moves through the tract, the systems of the present disclosure may use that position as a potential position for further review or anomaly detection. The temperature sensors of the present disclosure may include those sensors that are capable of measuring a temperature difference upon contact with a surface. In particular, some temperature sensors may have a resolution of less than 0.5° C. For example, the temperature sensors may be a thermistor, a resistance temperature type detector, or a thermocouple (e.g., type T thermocouple). Devices leveraging these sensors may be used, for example, during colonoscopies, on ingestible cameras, for diagnostic purposes, and/or for algorithm training purposes.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals), and others.

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments and, optionally, in combination of any embodiment described above or below, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof.

One or more aspects may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that actually make the logic or processor.

Some embodiments of the system include a computer implement method to train Artificial Intelligence (AI, which, as used herein, may include any type of machine learning) to recognize any combination of images and data from the far-infrared sensors as being above a threshold value for identification of an abnormality. Some embodiments of the system include at least a portion of one or more computer that include one or more processors and one or more non-transitory computer readable media with instructions stored thereon for implementing the at least a portion of each the embodiments described herein. In some embodiments, the non-transitory computer readable media includes instructions to implement the method to train AI to recognize any combination of images and text (as used herein, “text” includes any single or combination of one or more characters including letters, numbers linguistic symbols, and/or words) to identify threshold values for identification of abnormalities. In some embodiments, the system comprises independently operated AI such as AI for interpreting camera images, AI for interpreting the readouts from the plurality of far-infrared sensors (or a portion thereof), and AI for combining the information received from both data acquisition sets.

The image data may be processed by capturing regular images by the camera while the endoscope travels through a human gastrointestinal (GI) tract. The regular images may be placed together to determine any missed or insufficiently imaged area in a section of the human GI tract already travelled by the endoscope. If any missed or insufficiently imaged area is detected, information regarding any missed or insufficiently imaged area may be provided (e.g., by repositioning the camera to acquire the image, by supplementing the missed or insufficiently imaged data with thermal data from the plurality of IR sensors and/or temperature sensors). In some embodiments, missing images may be cross correlated and identified based on the measurements received from the plurality of far-infrared sensors. For example, if an abnormality is detected by the data taken from plurality of far-IR sensors, but not the camera, the distal tip may be repositioned to acquire camera image data of the area having the abnormal temperature. In some embodiments, the distinction in abnormality detection between the far-IR sensors and the camera may be sent to the endoscope operator who will reposition the distal tip. In some embodiments, the distinction in abnormality detection will cause an automatic repositioning of the distal tip.

The method may further comprise receiving structured light images associated with the regular images from the camera and deriving distance information of the regular images based on the structured light images, where the structured light images are captured by the camera while the endoscope travels through the human GI tract. Regular images may be normalized according to the distance information of the regular images and optical magnification information to facilitate the compiling of the regular images. The distance information may be used to determine whether a target area in one regular image is out of focus or in focus and if the target area is out of focus in all regular images covering the target area, the target area is determined as one missed or insufficiently imaged area.

In some embodiments, the endoscope or device for attachment to an endoscope may further comprise a motion sensing device to measure tube motion inside the human GI tract. For example, the motion sensing device may be an accelerometer, or a gyrator configured to transmit data to at least computer readable storage medium. The motion sensing device can be used to determine tube movement, tube trajectory, tube orientation or any combination thereof. These parameters may be used in the anomaly detection analysis. For example, increased movement speeds may correlate with increased slopes on heat data acquired from a single sensor. Integration of these multiple components as taken from independently the plurality of far-infrared sensors and/or camera may be used identify appropriate threshold values for anomaly detection (e.g., via the machine learning algorithms employed).

The endoscope may also be equipped with structured lights to capture regular images along with structured light images (SLIs). Thes SLIs can be used to derive the distance of the different areas in the image so that the stitching can be more accurate by using the 3D information of the imaged areas. In U.S. Pat. No. 9,936,151, which is being incorporated by reference in its entirety, a camera equipped with a regular light and structured light is disclosed to capture both regular images and structured light images using the same image sensor. The structured light images and corresponding regular images are captured temporally close so that the motion in between is expected to be small. Accordingly, the distance information derived from the structured light image may correlate with the regular image. Details regarding the camera design with SLI capture capability are disclosed in U.S. Pat. No. 9,936,151, which are hereby incorporated by reference. In certain implementations, the structured light is created at a sufficient wavelength to reflect off the tissue surface and be detected by at least one of the plurality of IR sensors.

The endoscope system of the present disclosure may evaluate an abnormality such as a lesion of a living tissue inside an organ. The endoscope system may include:
an endoscope configured to image a living tissue in an organ,
a plurality of far-infrared sensors to derive heat data from the organ, and
a processor that includes an evaluation unit configured to process a plurality of captured images and the heat data of the living tissue to evaluate and/or identify the presence of an abnormality and/or to evaluate and/or identify the abnormality (e.g., type of abnormality) in the organ.

For example, the evaluation unit may include one or more of:
an image evaluation value calculation unit configured to calculate an image evaluation value indicating an intensity of abnormality of the living tissue in each of the images from the pixel evaluation value for each of the plurality of images of the living tissue,
heat value calculation unit configured to calculate heat intensity abnormality indicating an intensity of abnormality of the living tissue in each of the images from a deviation (e.g., increase) of heat and/or temperature as compared to baseline or a thermal threshold value,
an imaging position information acquisition unit configured to acquire information of an imaging position inside the organ in which each of the images is captured in association with each of the images,
an abnormality position calculation unit that determines presence or absence of the abnormality in each of the images based and/or thermal deviations on whether the image evaluation value exceeds a predetermined threshold and/or the thermal threshold value,
a data correlation calculation unit configured to compare results from the image evaluation unit and the heat value calculation unit so as to obtain a start position and an end position of a region of an abnormality, and
an organ lesion evaluation unit configured to set a length of the lesion portion to be evaluated from the start position and the end position as extent information of the abnormality portion and evaluate the degree of abnormality in the organ using the position and extent information and a representative value of the image evaluation value of a captured abnormality portion image obtained by imaging the lesion portion.
   The abnormality position calculation unit may use, when the abnormalities exist at a plurality of locations, a location having a maximum length in the depth direction in which the lesion is continuously spread as the lesion portion to be evaluated. The length may be calculated based on a deviation in the temperature data, optionally in conjunction with one or more fields of view of the far-infrared sensors and/or the movement speed of the endoscope.

Systems for training artificial intelligence to recognize the presence of abnormalities are provided as well. These systems may comprise:
one or more computers comprising one or more processors and one or more non-transitory computer readable media, the one or more non-transitory computer readable media comprising instructions stored thereon that when executed cause the one or more computers to: by the one or more processors, import far-infrared sensor data and optionally an image from an endoscope of the present disclosure camera by the one or more processors, wherein the processors may be configured to
identify one or more abnormalities based on a threshold image data (e.g., as captured from the camera),
identify one or more abnormalities based on threshold heat data (e.g., as captured from the plurality of far-IR sensors and/or temperature sensors),
compare heat data and image data to identify if both systems return a positive identification, and
rebalance the threshold value based on positive or negative correlations in the comparison.
   In various implementations, the rebalancing of the threshold value may involve positive identification of abnormalities by, for example, a practitioner performing an endoscopy. The machine-learning may be trained to recognize the presence of abnormalities by supervised learning, unsupervised learning, reinforcement learning, and/or ensemble learning. The algorithms leveraged by the artificial intelligence may include linear, logistic, K-means, anomaly detection such as isolation forest, neural net (particularly for reinforcement learning), KNN, decision tree, random forest, SVM, naïve Bayes, or combinations thereof. In some embodiments, the machine learning algorithm (e.g., anomaly detection such as isolation forest) is chosen to provide a percentage of outliers (or false positive rate) of greater than 20% (e.g., greater than 30%, greater than 40%, greater than 50%, from 20% to 60%). The machine learning algorithm (and corresponding real time analysis) may be chosen based on the detection rate of the far-IR sensors in the device (e.g., each far-IR sensor may have a detection rate of at least 1 Hz (e.g., at least 2 Hz, at least 3 Hz, at least 4 Hz, at least 5 Hz, at least 6 Hz, at least 7 Hz, at least 8 Hz, at least 9 Hz, at least 10 Hz, from 2 Hz to 20 Hz, from 3 Hz to 20 Hz, from 4 Hz to 20 Hz, from 5 Hz to 20 Hz, from 6 Hz to 20 Hz, from 7 Hz to 20 Hz, from 8 Hz to 20 Hz, from 9 Hz to 20 Hz, from 10 Hz to 20 Hz)). In some embodiments, multiple machine-learning algorithms may be utilized (e.g., in devices with different sensors having, for example, different detection frequencies).

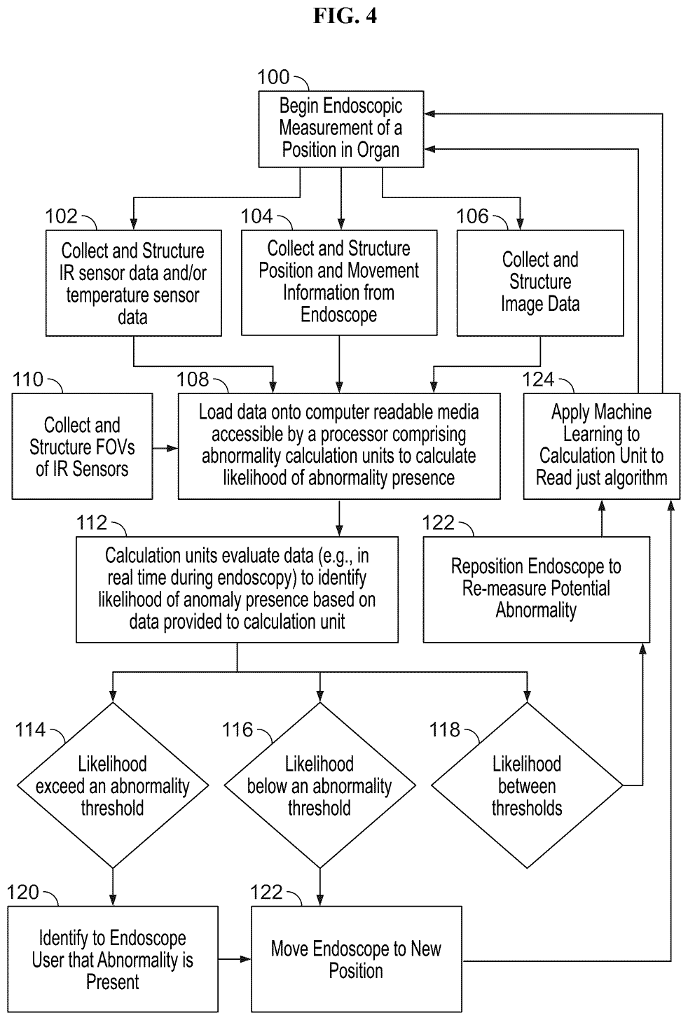

The machine learning algorithm may interpret the raw data output by the plurality of sensors in real time to identify abnormalities as the distal tip of the endoscope passes through various positions in the organ or space being examined. An exemplary flow chart for an endoscopic procedure and system using the plurality of far-IR sensors described herein is provided in FIG. 4. At step 100, the measurement procedure may begin. Step 100 may include any position of the distal tip as it moves through the organ or tissue. Step 100 may for example, be started by an operator, started at a point during the procedure (e.g., as evidenced by some visual marker), or be started as soon as a baseline ambient temperature of the tissue has been established (e.g., as measured by one or more far-IR sensors and/or temperature sensors). In some embodiments, each “position” may include an aggregation of several measurements from each far-IR sensor. For example, a position may be considered an aggregation of data from at least one far-IR sensor of from 1 to 100×the detection rate. For example, if the calculation unit is processing data from a far-IR sensor having a detection rate of from 1 to 10 Hz, each position may include a measurement of from 0.1 s to 100 s (e.g., from 0.1 s to 50 s, from 0.1 s to 20 s, from 0.1 to 10 s, from 1 s to 10 s).

The system may collect and structure the far-IR sensor data at step 102. In some embodiments, the system acquires the far-IR sensor data from the far-IR sensors directly and transmits the raw data to a readable medium accessible by a processor comprising instructions for abnormality detection at step 108. In some embodiments, the data (particularly data from multiple sensors and, multiple nonidentical sensors having, for example, different fields of view and/or detection rates) is collected and structured to be easily analyzed by the processor at step 108. Optionally, the endoscope may also collect and structure position information of the distal tip such as movement speed (e.g., as measured by an accelerometer) and rotation at step 104. In some embodiments, the system may also optionally collect and structure image data from the endoscope camera at step 106. Step 102 and optional steps 104 and 106 may each be transmitted to the processor at step 108 for analysis, interpretation, and identification of abnormality positions. In some embodiments, the fields of view of the plurality of infrared sensors and, optionally camera, are collected and sent to the processor for analysis of the real time data at step 110. In some embodiments, the calculation unit has been trained on a specific geometry of the plurality of the far-infrared sensors and, optionally, camera, and step 110 is not required. The calculation units, by artificial intelligence and/or machine-learning, then interpret the data sent to the processor at step 112 to identify the likelihood of an abnormality presence in the endoscope position. The calculation unit identifies if the endoscope position has a high likelihood of an abnormality (114) (e.g., the calculation units detect a false positive rate of less than (or from 0.1%) 20% such as when there is a large deviation in far-IR signal across several subsequent measurements and positions), a low likelihood of an abnormality (116) (e.g., the calculation unit detects a false positive rate of more than (or to 100%) of 80%, or an indeterminate likelihood (118) (e.g., a false positive rate of from 20% to 80%).

If the likelihood is high (114), the system may notify the endoscope user that an abnormality is present at step (120). For example, the calculation unit calculate and overlay far-IR sensor data over optical camera images for depiction on the user's screen, a notification may be received of an unviewed anomaly and, optionally, provide options to the user such as to rinse the area of an unviewed anomaly and to reposition the camera for additional viewing of the abnormality (e.g., following rinse of the abnormality) such as by orienting another set of sensors and/or the camera to view the abnormality or to another location for continuation of the endoscopic procedure at step 122. If the likelihood is low (116), the endoscope is moved to the next position for study at step 122. If there is a likelihood between the thresholds, the endoscope 122 may be repositioned to retake the measurement and remeasure the potential abnormality at step 122. This remeasurement may involve a measurement of the same parameters in the previous measurement or involve a new measurement of the tissue (e.g., by overlapping the field of view of a different set of far-IR sensors and/or the camera with the potential anomaly, by rinsing the area to identify potential obstructions prior to measurement). This data may be sent to a processor configured with machine learning and/or an artificial intelligence algorithm at optional step 124 to allow for real time learning from these possible events, and subsequent integration into the calculation (which may occur during or after the endoscopic procedure). If, for example, repeated measurements are made over the same position, with a final positive identification, the calculation unit may integrate this information to allow for similar data situations to be associated with an abnormality. The system is then ready to proceed with measurement of a new position of the organ or space. In some embodiments, based on the abnormality, a treatment may be performed. For example, the endoscope (as controlled by the user) may cut and/or collect some or all of the tissue (e.g., for further analysis following the endoscopy, to remove the abnormality). In some embodiments, the endoscope (as controlled by the user) may mark the tissue, for example, by injecting a substance such as a fluorescent or chemiluminescent substance into tissue at the abnormality location, for the location to be found at a later time, or administer a substance to help treat the abnormality.

The calculation units may leverage algorithms developed by machine-learning or artificial intelligence. In particular, in embodiments where the calculation unit involves optical images and far-IR sensor data artificial intelligence is used (e.g., to correlate far-IR abnormalities with position on the optical image). The artificial intelligence may be a neural network (e.g., deep learning, deep convolutional, or recurrent neural network) comprising a series of neurons. A neuron is an architectural element used in data processing and artificial intelligence, particularly machine learning on the weights of inputs (e.g., data from one or more far-IR sensors and their corresponding geometry on the distal tip, the optical image) provided to the given neuron. Each of the neurons used herein may be configured to accept a predefined number of inputs from other neurons in the neural network to provide relational and sub-relational outputs for the content of the optical images being analyzed in concert with the far-IR sensor data. Individual neurons may be chained together and/or organized in various configurations of neural networks to provide interactions and relationship learning modeling for how each of the optical images and far-IR sensor data are related to one another.

For example, a neural network node serving as a neuron may include several gates to handle input vectors (e.g., sections of an image, a vector of far-IR sensor data, a structured array of data from several far-IR sensors, the geometry of the far-IR sensors), a memory cell, and an output vector (e.g., contextual representation for display to the endoscope user or storage for subsequent examination). The input gate and output gate typically control the information flowing into and out of the memory cell, respectively. Weights and bias vectors for the various gates may be adjusted over the course of a training phase, and once the training phase is complete, those weights and biases may be finalized for normal operation (or tuned during operation based on continued training. These neurons and neural networks may be constructed programmatically (e.g., via software instructions) or via specialized hardware linking each neuron to form the neural network.

The neural network may utilize features for analyzing the data to generate assessments (e.g., patterns in an image and/or far-IR sensor data). These features may be an individual measurable property of a phenomenon being observed (e.g., an increase of temperature of an abnormality, a change in the surface of the tissue). The concept of feature is related to that of an explanatory variable often used in statistical techniques such as linear regression. Further, deep features represent the output of nodes in hidden layers of the deep neural network which may be identified during the artificial intelligence training.

Control commands of the endoscope may also be generated based on computer analysis of data that is collected by the far-IR sensors. The computer analysis may include use of AI/ML techniques for more accurate and precise control command generation and determination of whether the endoscope (or device attached thereto) is operating according to expected conditions/parameters. For example, over time, data can be collected about force and speed or other time information of the distal tip. This data can be used to train one or more models to accurately and precisely generate control commands the distal tip during an operation (e.g., such as provide repositioning, rinse, surgical options).

The one or more models described herein can be any type of machine learning model, including but not limited to correlation models, ordinary least squares, convolutional neural networks (CNNs), or supervised machine learning. Different models can be generated for different types of procedures/operations. For example, artificial intelligence and machine-learning model may be trained by using a YOLO (e.g., YOLOv2) or Detectron algorithm. Different models can also be generated for different types of sensor and distal tip geometries or endoscopes having different functionalities. In some implementations, different models can also be generated for different patient profiles, demographics, or other patient information.

FIG. 5 provides an exemplary flow chart for the ML/AI training algorithms of the present disclosure utilizing the data afforded from the plurality of far-IR sensors and optionally, the endoscope camera. A training session typically begins (200) with the collection and structuring of data from one or more far-IR sensors at step 202 and the optional collection of data about the distal tip position (step 204) and camera images (step 206). This data is sent to the readable media accessible by a processor comprising the machine learning algorithm. In some embodiments, the geometry of the plurality of far-IR sensors is also provided to the readable media. This data may be taken from procedures (e,g., procedures where a positive correlation was directly made by a user between abnormalities and far-IR sensor data) and/or synthetic data produced to help train the algorithm based on analysis of smaller datasets. At step 212, the machine learning algorithm may interpret these data sets and identify variables to afford threshold detections of abnormalities at step 214. These variables may include a temperature differential from ambient as measured by the far-IR sensors, a temperature differential associated with certain endoscopic distal tip movements, a temperature differential correlated with optical images from the camera. Leveraging this information, the machine-learning algorithm may create a calculation unit for use in an endoscopic procedure. In some embodiments, the information from a calculation unit may be reused in a subsequent training session to further analyze a new set of data based on the previously ascertained data correlations. In some embodiments, the machine learning algorithm may learn during an endoscopic procedure and provide changes to the weights and correlations of abnormality detection in the calculation unit in real time.

### EXAMPLES

The following examples illustrate specific aspects of the instant description. The examples should not be construed as limiting, as the example merely provides specific understanding and practice of the embodiments and its various aspects.

### Example 1: Sensor Evaluation

Several far-infrared sensors were tested to identify their ability to stably detect a temperature at various distances within an anatomical range from a heat source. For the evaluations, a square black body radiator was placed in a test booth and set to produce a temperature 2° C. greater than ambient. The test booth was used in order to block ambient temperature variations and to mimic physiological conditions. Uniformity across the blackbody radiator was ensured using an IR camera.

Measurements from each sensor were compared to a thermocouple measurement. Sensors tested were Exceletis and Melexis sensors having a Field of View (FOV) of 120°, 90°, 50° 35°, and 5°. FIG. 6 shows the accuracy of tested sensors (as a percentage difference from the thermocouple measurement) for each sensor evaluated measured at 0° at distance from 0 to 1 inch from the blackbody radiator face.

A plastic mask having a 5 mm hole was placed over the blackbody radiator and allowed to heat to uniformity. The heater was set to 26.0° resulting in a uniform mask temperature of 24.1°.

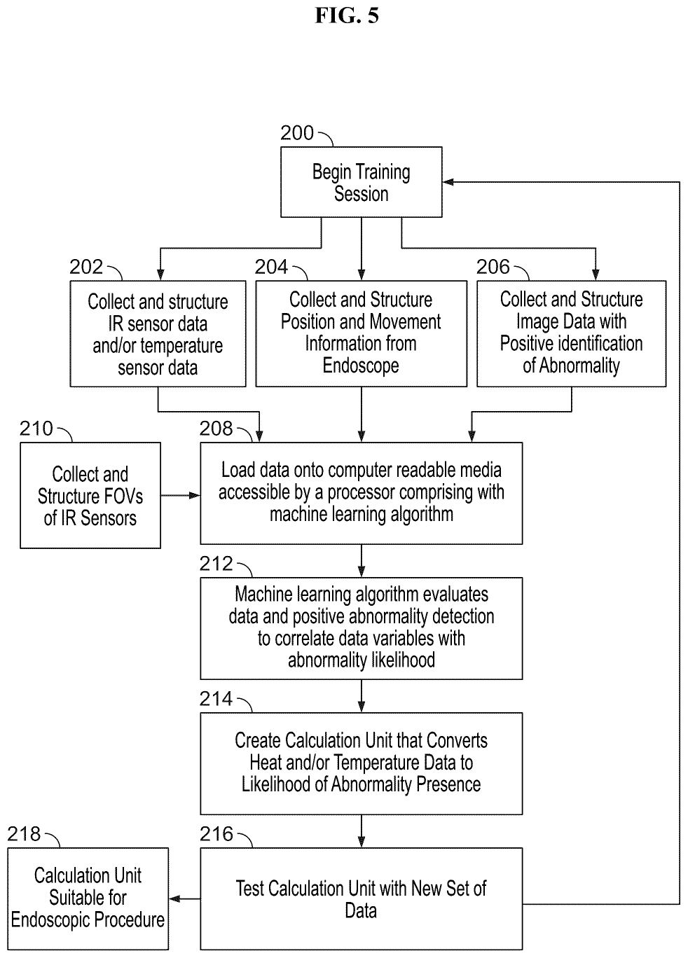

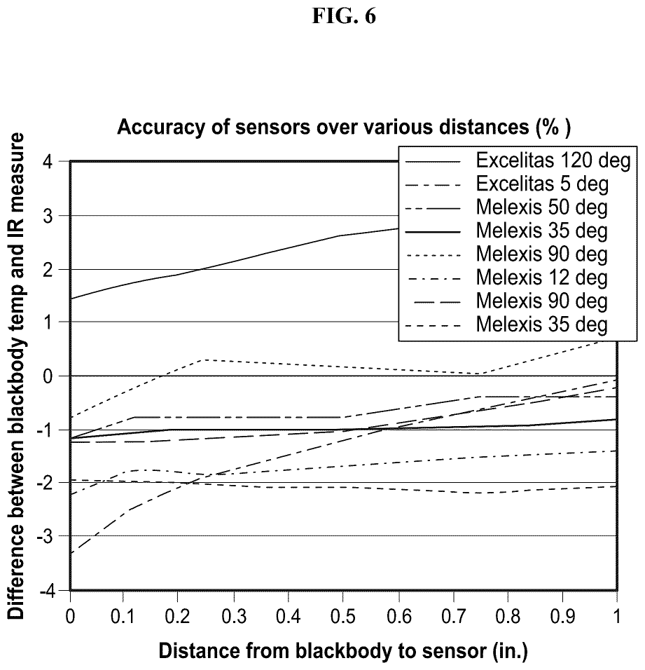

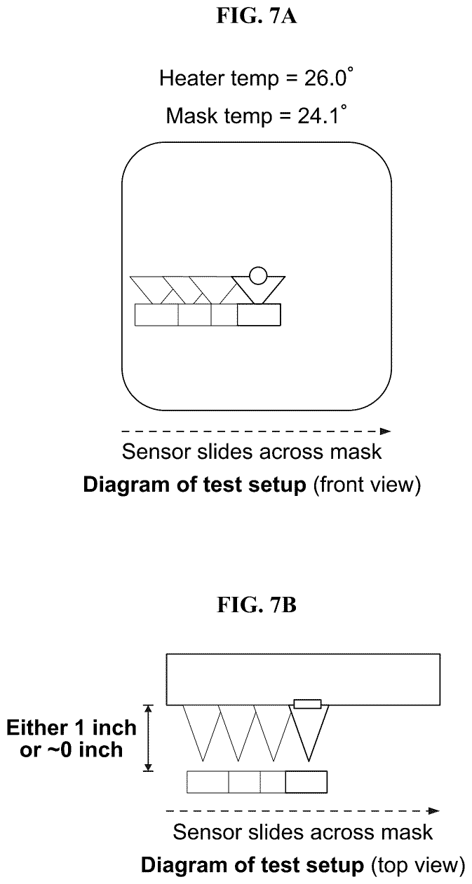

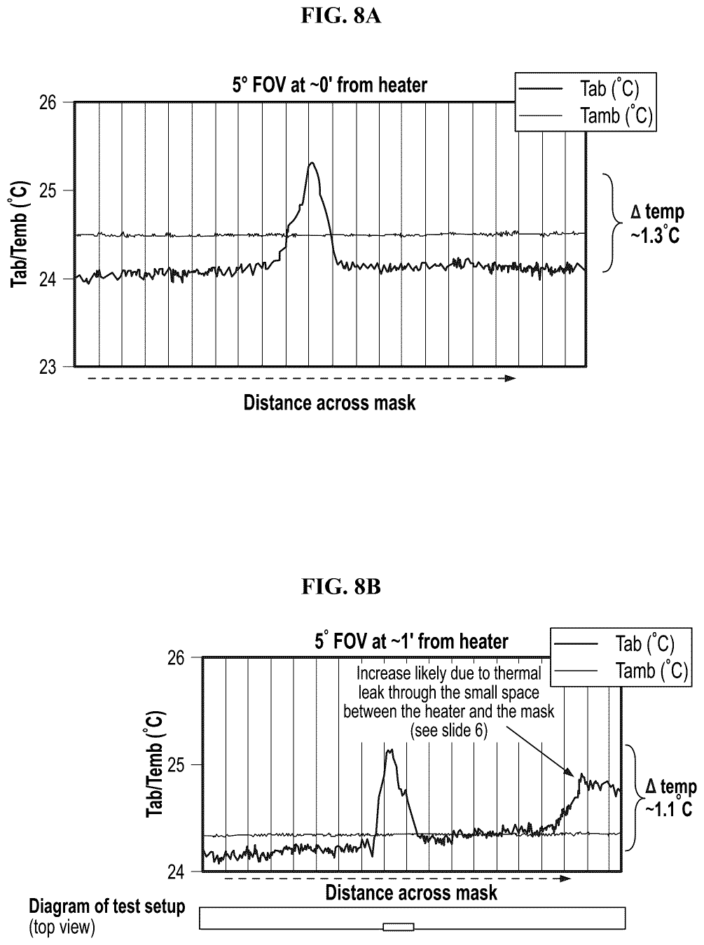

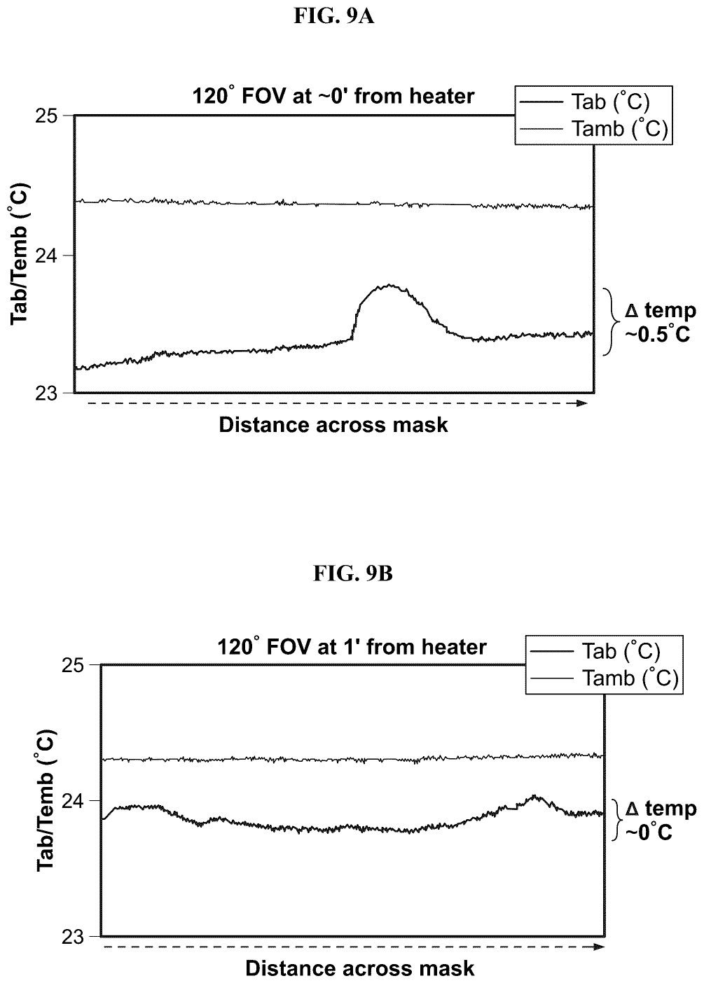

Several sensors were moved across the face of the mask and over the hole to identify the temperature fluctuations associated with the mask geometry. A diagram of this experimental protocol is shown in FIG. 7A and 7B. As the sensor is moved across the mask such that the hole is within the sensor FOV at some positions during movement. The temperature readings were monitored during movement. FIG. 8A shows the Excelitas 5° FOV sensor temperature readings when the is moved directly next to the mask (“0′ from heater”) and FIG. 8B shows the readings at 1 inch (“1′ from heater) separation from the mask. FIG. 9A shows the Excelitas 120° FOV sensor measurements when positioned at the mask and FIG. 9B shows the measurements with the sensor positioned 1″ away from the mask. These figures also provide the ambient temperature. As can be seen, as the sensor is moved across the hole, the increased temperature variation can be identified. However, the 120° FOV was unable to detect the thermal variation at 1″ away from the mask. FIG. 8B also has an artefact due to a thermal leak between the heater and the mask which caused a higher temperature to be measured.

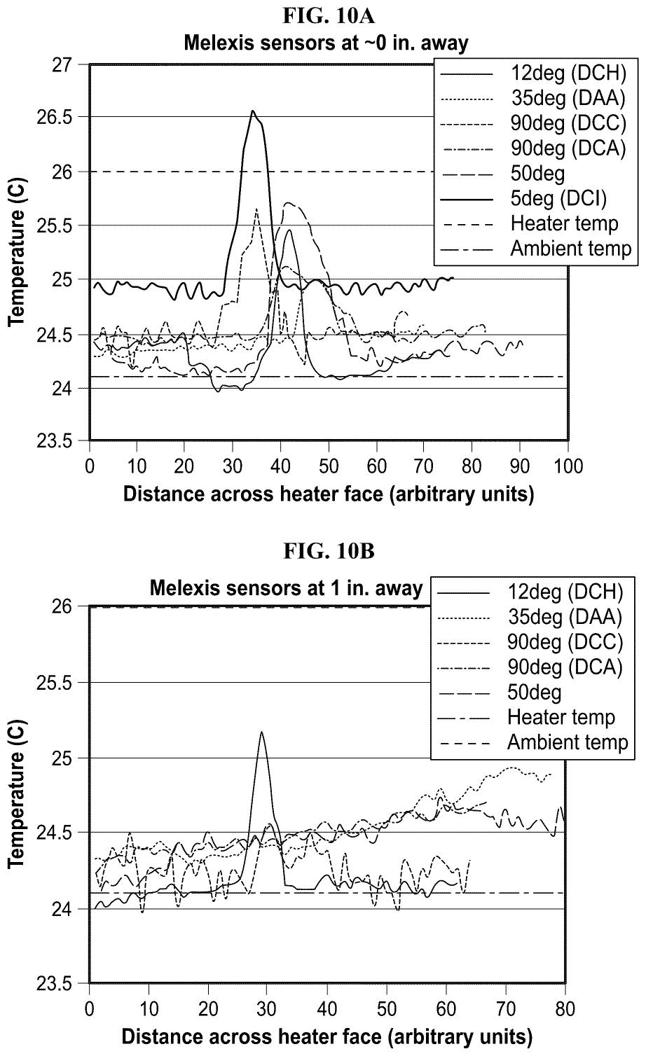

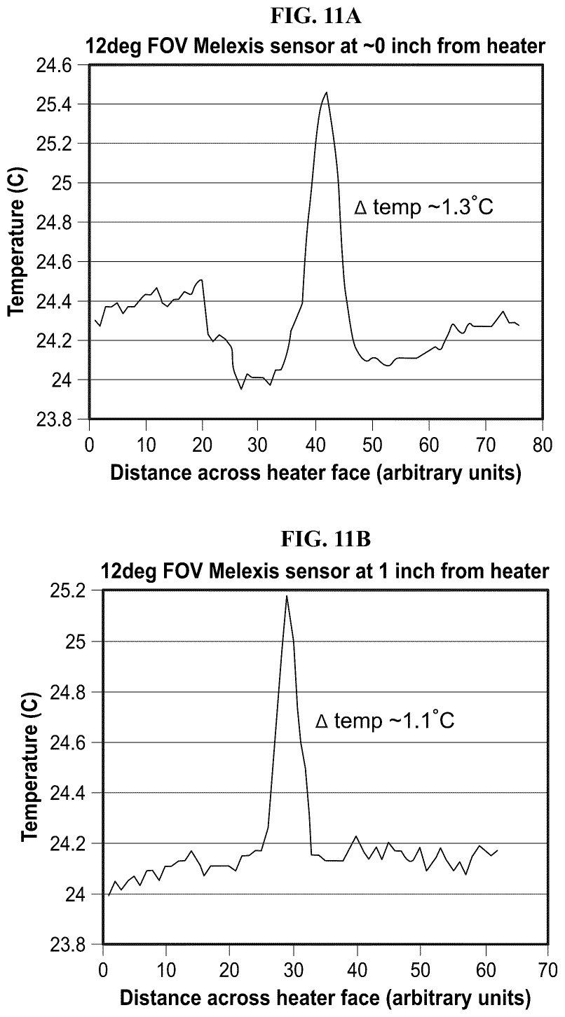

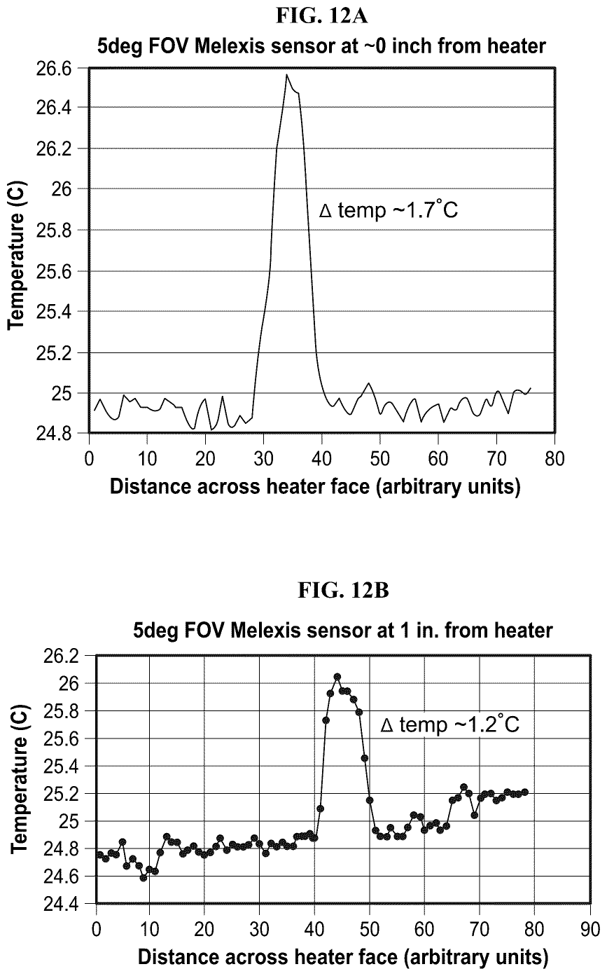

The Melexis sensors were also measured and measurements are shown in FIGS. 10A (0 inches from mask) and 10B (1 inch from mask). As can be seen, each sensor measures the mask differently at these positions, with measured temperature, position, and size of the hole being affected by changes in FOV and sensor type. FIGS. 11A-B provides similar measurements for the 12° FOV Melexis sensor and FIGS. 12A-B provides measurements for the 5° FOV Melexis sensor. These sensors were able to accurately and consistently identify hole shape and size at the anatomical limits studied.

### Example 2: Sensor Evaluation on Synthetic Bowels

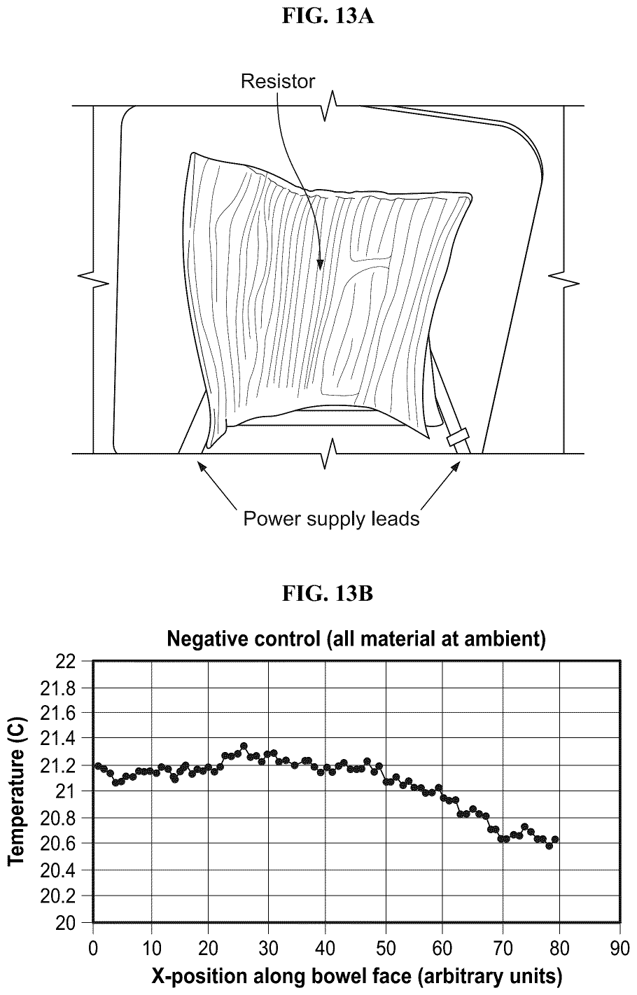

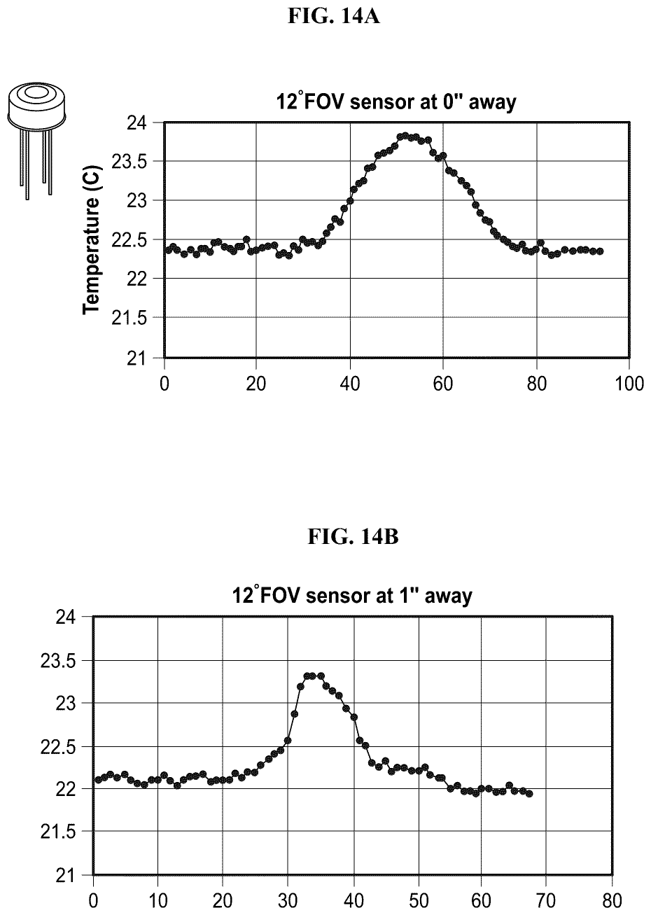

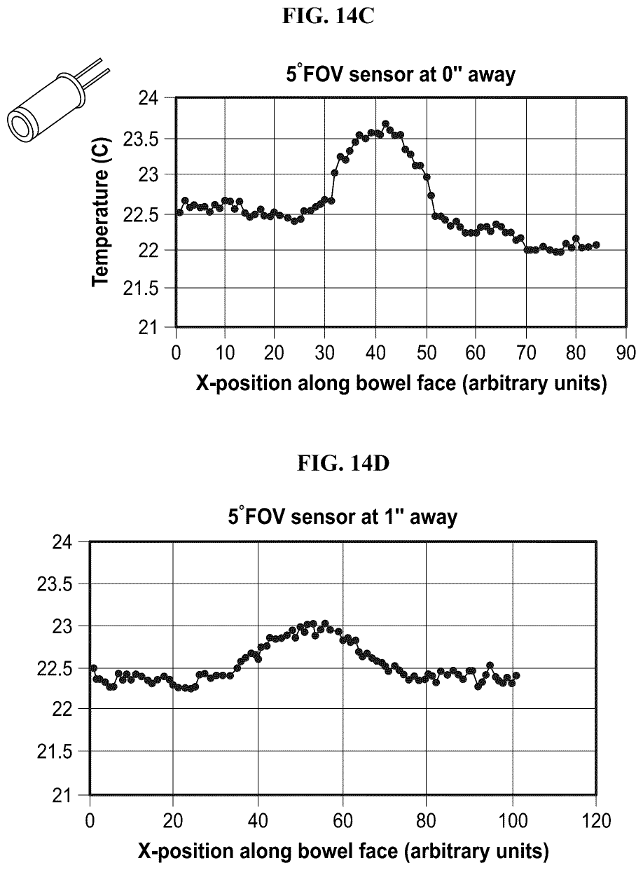

To simulate sub surface temperature measurements, a resistor was placed under synthetic bowl. A negative control experiment was performed where sensors were moved across the synthetic bowl when all temperatures were held at ambient temperature. An annotated image of the experimental setup showing the resistor position beneath the synthetic bowl and power supply connections thereto is shown in FIG. 13A and the measurements from the negative control are shown in FIG. 13B. FIGS. 14A-D provide the 5° FOV and 12° FOV sensor measurements as the sensor is positioned across the synthetic bowl at 0 inches and 1 inch from the mask. The ambient temperature for these measurements was 24.0° C. and the resistor temperature was to 26.0° C. The broader distribution as compared to the mask holes in Example 1 is likely due to thermal transmittance of the synthetic bowl as compared to plastic. Nevertheless, these sensors were able to identify thermal variations due to subsurface temperature fluctuations.

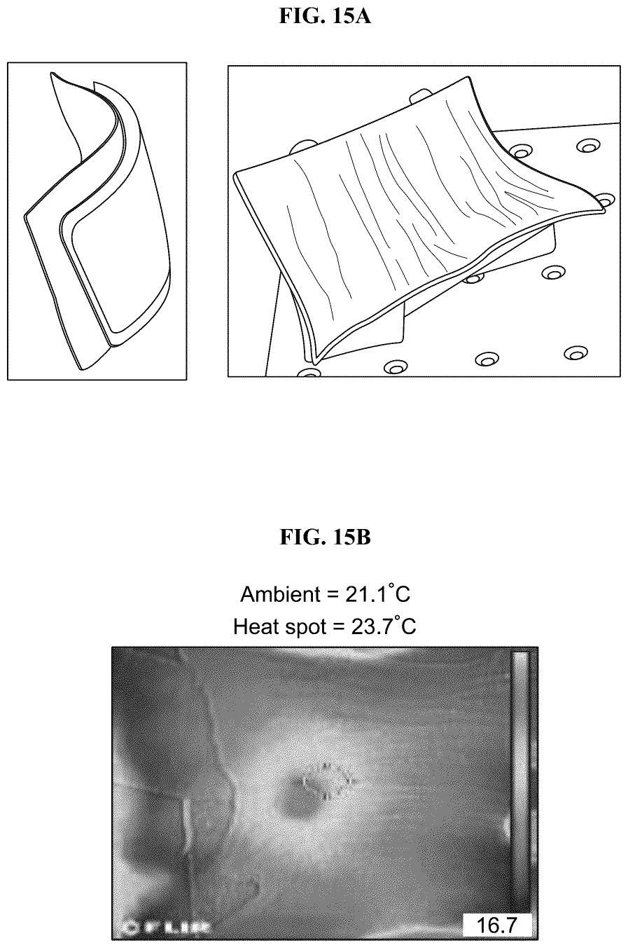

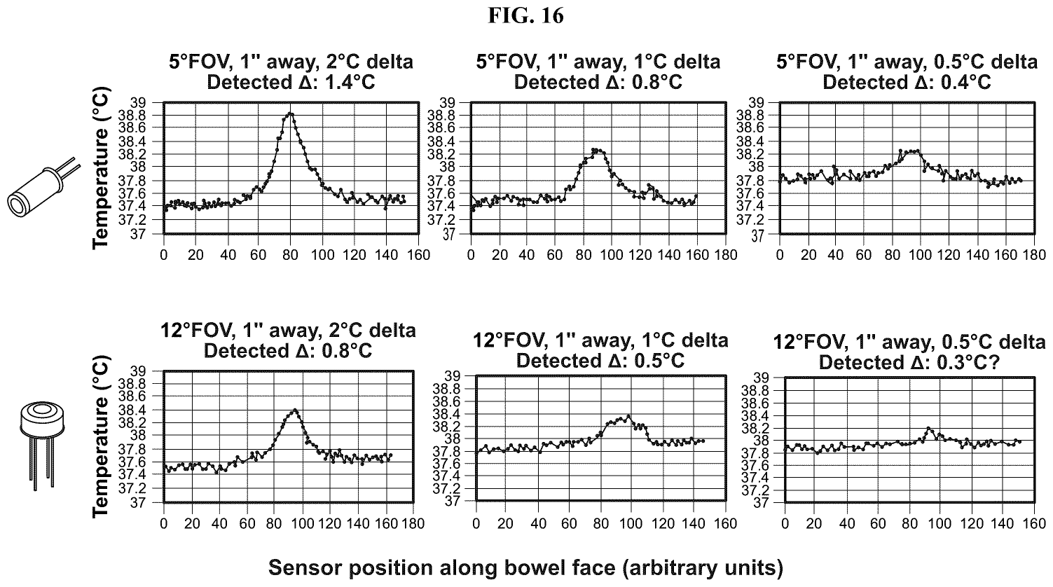

The synthetic bowel was oriented in a curved geometry in order to more closely mimic the GI tract as shown in FIG. 15A. A resistor was placed behind the curved synthetic bowel. A heat map as measured by the IR camera is shown in FIG. 15B. Sensor measurements were made along the longitudinal axis of the curvature and are shown in FIG. 16 for the 12° and 5° FOV sensors at variations positions from the curved synthetic bowels and at different resistor temperatures (as compared to ambient).

These measurements illustrate that only certain sensors are able to provide accurate thermal measurements over anatomical distances typically required for endoscopic measurement. In particular, the 5° and 12° FOV sensors were highly accurate over the distances measured, each providing some signal at all test conditions. Similar experimentation may be performed to identify and train machine learning algorithms to accurately identify the presence of abnormalities, and particularly, subsurface abnormalities.

### Example 3: Integration of Multiple Sensor Measurements

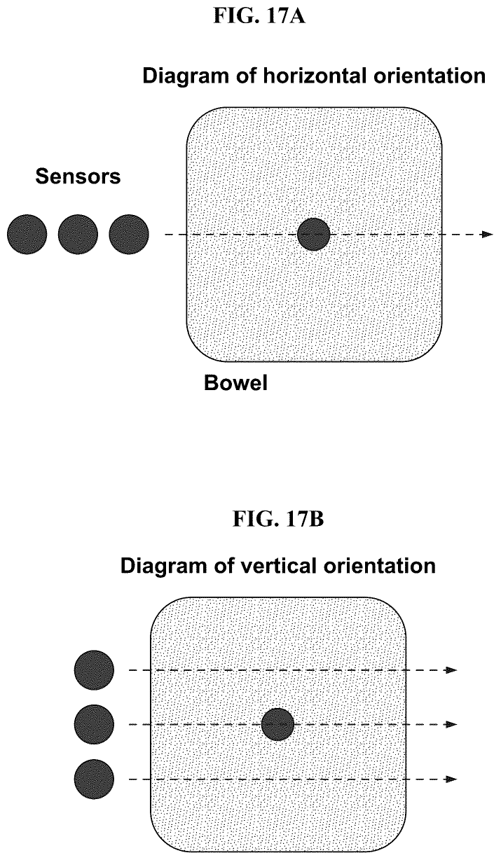

Three identical Melexis 5° FOV sensors were placed on a substrate and moved across a curved synthetic bowel having a resistor on one side to simulate a subcutaneous anomaly. The sensors were placed in a linear row on the substrate such that scans could be performed in a horizontal orientation (FIG. 17A) or vertical orientation (FIG. 17B) with respect to the anomaly. “Horizontal” and “vertical” in this context are in relation to the linear configuration of sensors relative to movement. A “horizonal” configuration may be considered to be similar to a circumferential arrangement of sensors around the tube and a “vertical” configuration may be considered to be similar to a linear arrangement of sensors along the major longitudinal axis of the tube.

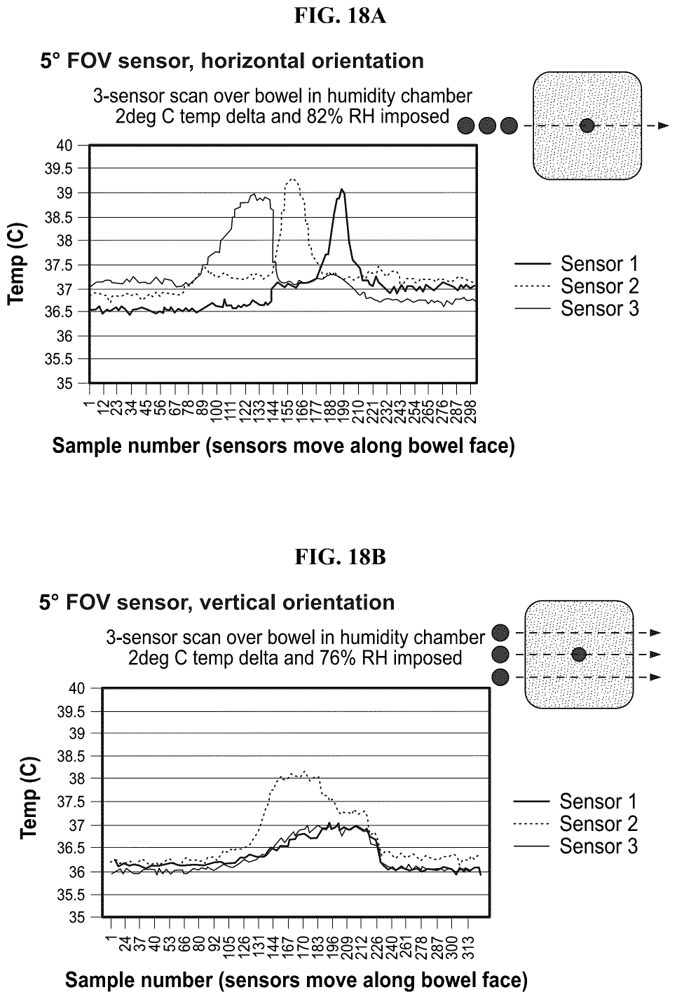

Thermal data was collected for sensors arranged in each orientation and is shown in FIGS. 18A (horizontal orientation) and 18B (vertical) orientation. As the three sensors pass over the abnormality in the horizontal location, the abnormality can be measured, albeit at different positions of the substrate as a whole. In contrast, as the three sensors pass over the abnormality in the vertical location, the width of temperature fluctuations is identified. However, the central censor (Sensor 2) shows the higher thermal variation as compared to the edge sensors (Sensors 1 and 3) which register an identical response.

### Example 4: Animal Study

Three custom fixtures were designed and used to collect data during an animal study. This study was designed to determine if FAR-IR sensors could detect a simulated heat spot two degrees Celsius warmer than its surroundings in live animal tissue consistently and in multiple locations. Additionally, these studies were designed to elucidate sensor performance in conditions representative of a colonoscopy. For example, the studies may evaluate:
1) use of the IR sensor to identify a simulated polyp at a fixed distance of 0.1″ (0.254 cm), a distance chosen to eliminate variability that may arise in the experiment due to any FAR-IR proximity dependence;
2) use multiple sensors with different fields of view (FOV);
3) controlled blind study to identify a heat source by observation of FAR-IR data on a graphical user interface (GUI) to identify the beginning and end of a 2° C. temperature difference (Δ or detlta) without direct visualization of the animal, fixture, or operator, while fixture with sensor is travelling a controlled path; and
4) assess sensor function following application of a stressor that may be encountered during a colonoscopy including an intentional bowel surface rinse with 0.9% saline, with fecal matter disposed on the sensor; and with air/CO₂ insufflation environment;

Three custom fixtures were designed and used to collect data during the animal study.

### Sensor Fixture And Design

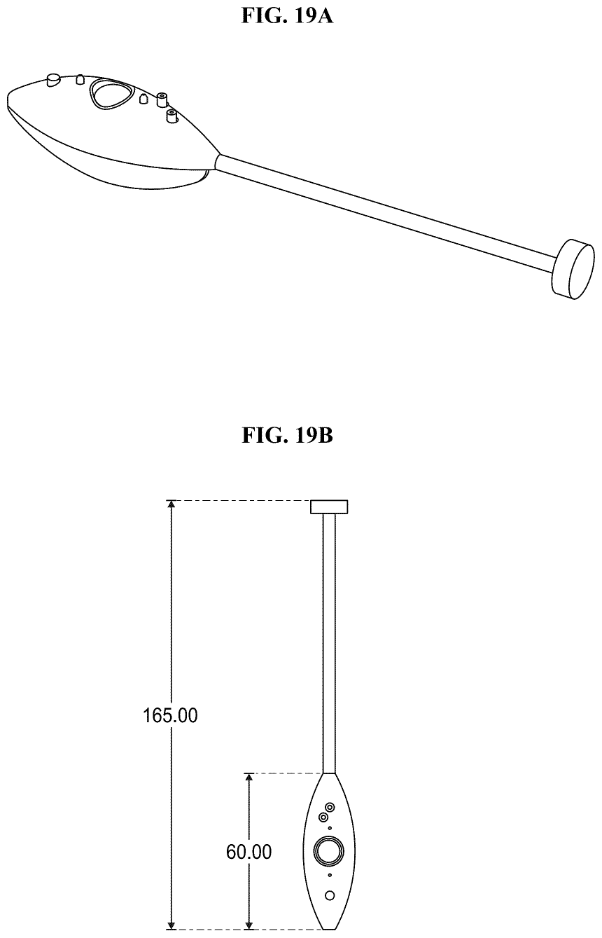

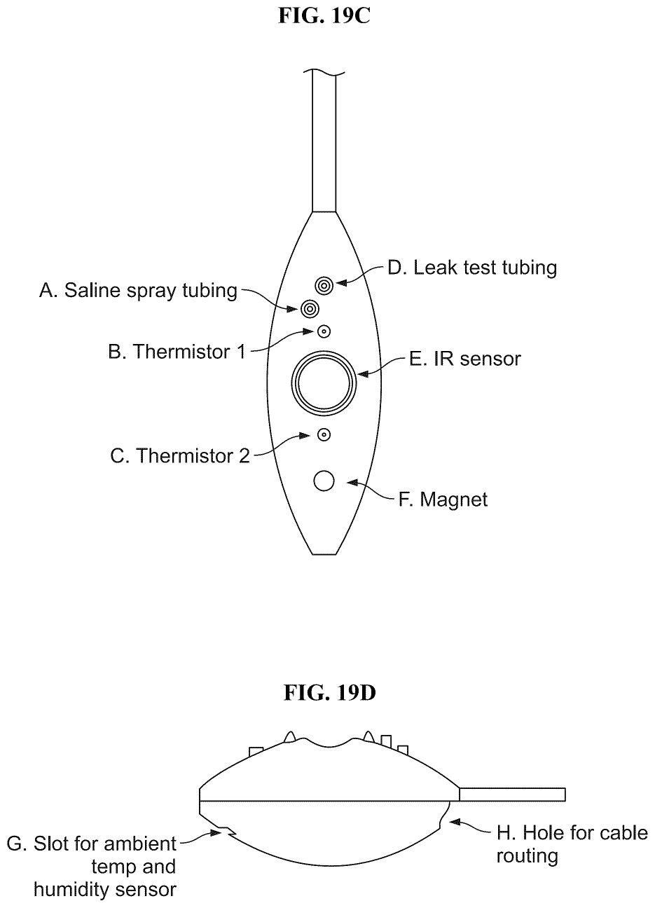

A housing for the sensor array was designed, assembled, and leak tested prior to the animal study. This housing held the sensors in fixed and known locations and offered protection from the live tissue environment. The sensor fixture was designed in SolidWorks and fabricated with FormLabs Form3, 3D printer in Clear resin. UV-cure glue was used to affix the hardware into the housing and to seal the housing. A perspective view and cross section view of the housing are provided in FIGS. 19A and B, respectively.

A top and side view of the sensor housing is provided in FIGS. 19C (top) and D (side) identifying the relative locations of the saline spray tubing, the leak test tubing, each thermistor (Thermistor 1 and Thermistor 2), the FAR-IR sensor array, magnet, position for temperature and humidity sensors, and hole for cable routing.

The saline spray tubing was used to apply saline to the bowel wall from a syringe outside the bowel.

Thermistor 1 protruded slightly above the surface of the housing to ensure good contact with the tissue when sliding within the colon. This thermistor reads the surface temperature of the tissue such that at the beginning of each test in a new location, Thermistor 1 is placed directly beneath the heat source (a resistor attached to the exterior of the bowel) to allow for the temperature of the heat spot to be set and adjusted to the appropriate temperature above the baseline tissue temperature. Thermistor 2 serves the same purpose as Thermistor 1, but measures the baseline tissue temperature. The difference (or A between Thermistor 1 and Thermistor 2) is the temperature difference being investigated in any experiment. The magnet was used to align the heat source with Thermistor 1 in the position below the resistor contact point.

During experimentation, the leak test tubing connected to a pressure gauge and syringe in order to identify leaks in the housing.

Two types of FAR-IR sensors were assessed: a 5° Field of view (FOV) sensor (Melexis part number MLX90614ESF-CDI-000-TU) and a 12° FOV (Melexis part number MLX90614ESF-CDH-000-TU).

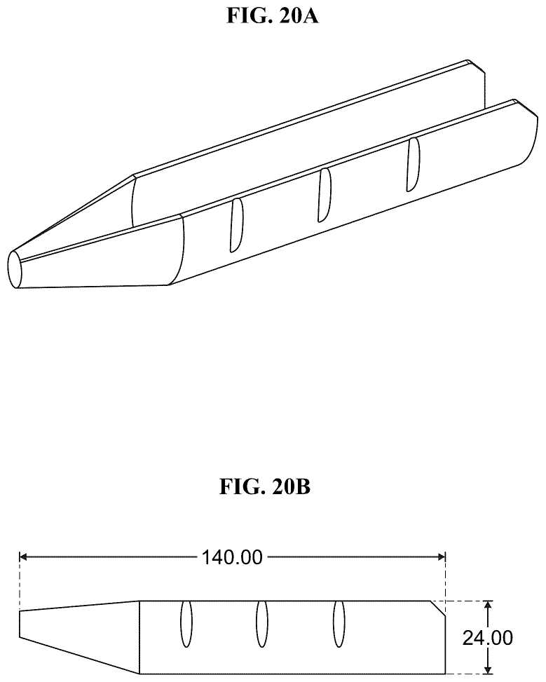

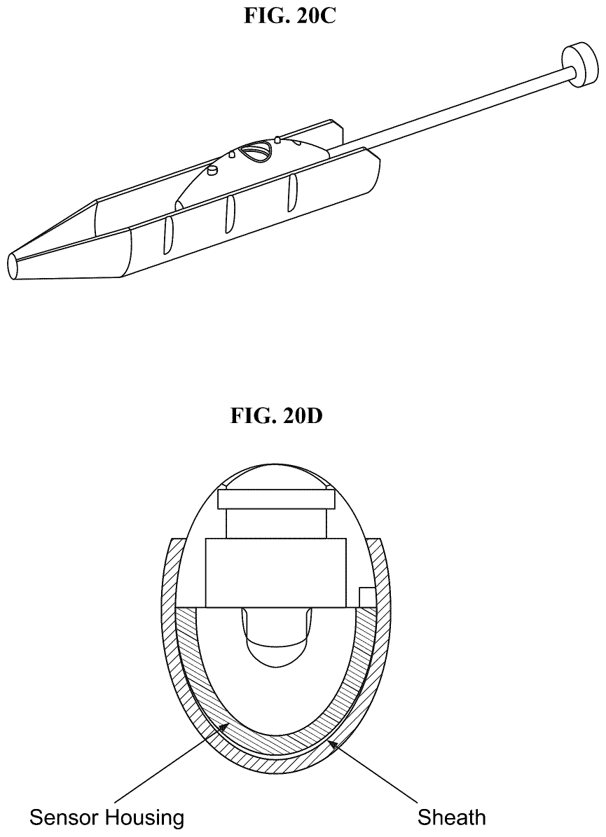

A sheath was developed for insertion into the colon to guide the sensor fixture during travel in the colon during experimentation. A perspective view and cross-sectional view are provided as FIGS. 20A and B, respectively. FIG. 20C provides a perspective view of the housing and sensor positioned in the sheath and FIG. 20D provides a front view of the housing and sensor in the sheath. The sheath restricts the housing's freedom to rotate about its long axis due to the elliptical cross section.

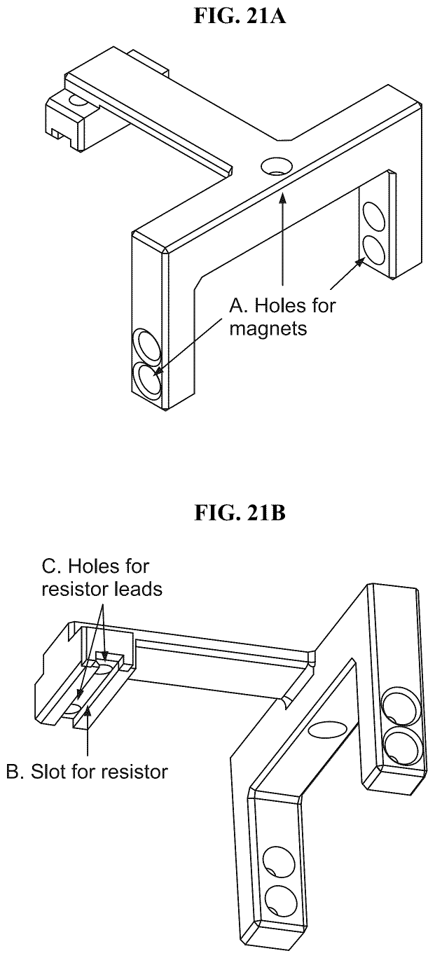

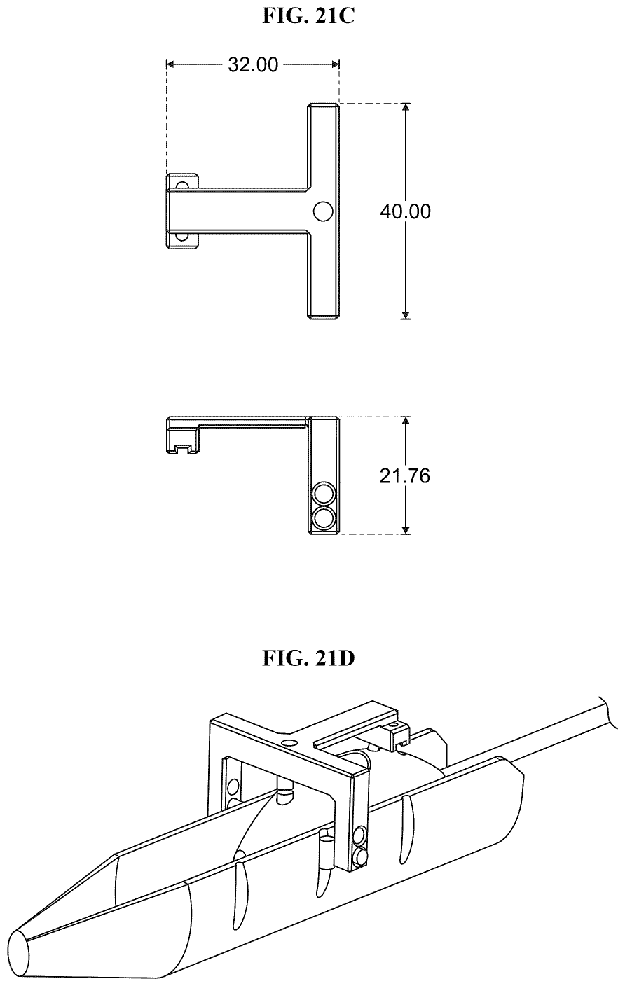

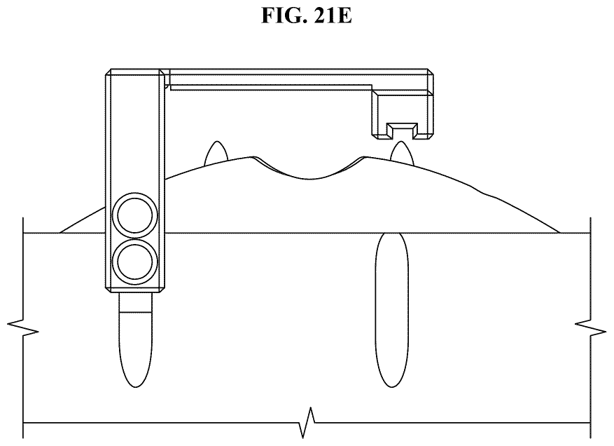

A resistor holder was also developed in order to position the resistor to a known location on the exterior of the bowel. The fixture held the resistor in a known location with respect to the sheath, housing, and sensor location. Magnets were glued onto the sides of the sheath and connect to the magnets on the arms of the resistor figure as shown in FIGS. 21A-E. This attachment centers the resistor in the sheath during experimentation through the attractive magnetic fields between the sheath magnets and the attachment magnets. When a magnet is inserted into the top hole on the attachment in order to align the housing via the magnet thereon, Thermistor 1 is considered aligned with the resistor. In this configuration, Thermistor 1 obtains an accurate reading of the heat spot produced from the resistive heater thus affording a direct measurement of the heat spot as compared to the surrounding tissue (Thermistor 2). By alteration of the voltage parameters to the resistor, the heat spot can be set to a desired temperature difference from the surrounding tissue and FAR-IR measurements can be obtained as the housing and sensors are able to move freely within the sheath.

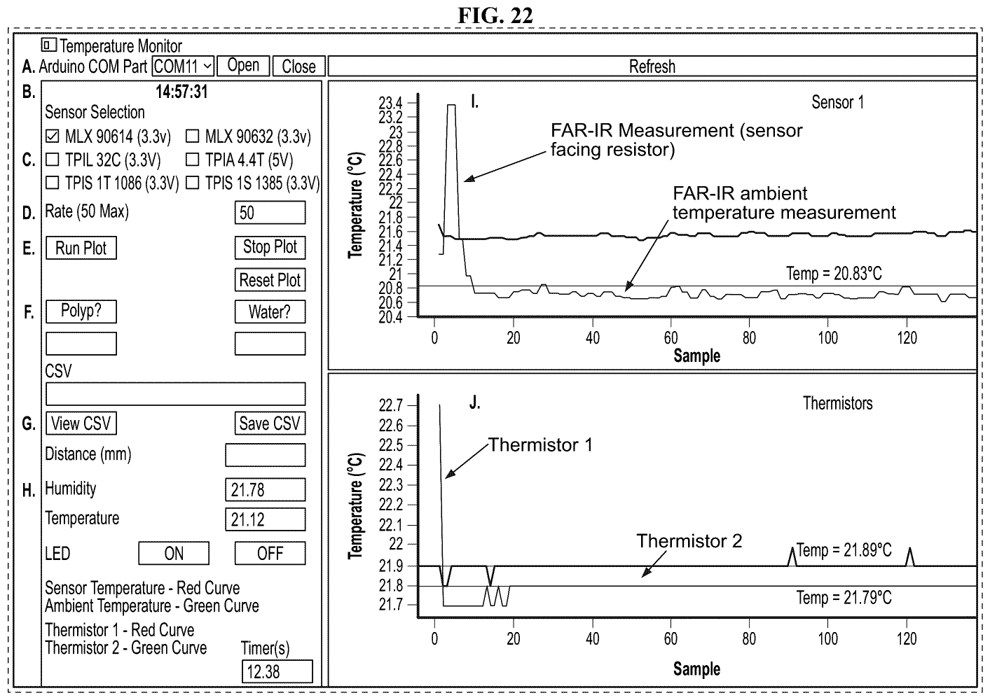

Sensor data was transmitted through a custom-built printed circuit board (PCB) connected via USB connection to a laptop where a custom graphical user interface was configured to display all sensor data in real time. The GUI also allowed the operators to mark specific events in the data file such as polyp presence or the use of saline spray. A screenshot of the GUI is provided as FIG. 22 including: A) an indicator for board connection, B) a clock for notation of specific time events by the operator, C) options for the selection of various sensors, D) an input for the sampling rate of the sensor tested, E) options for beginning or stoppings measurements or plotting, F) ability to mark data for polyp or saline detection, G) filename settings, H) sensor readouts such as ambient humidity and temperature, I) plot of FAR-IR data being measured, and J) plot of the thermistor data. In these exemplary traces, the FAR-IR sensor measurements can be seen to match the thermistor readings.

### Procedure

A juvenile pig was put under anesthesia and its abdomen was incised to expose the gastrointestinal tract within the abdominal cavity. The spiral large intestine, with blood supply intact, was lifted from the abdominal cavity and exposed for access. Nine different locations of the large intestine were tested. For each location in the large intestine, an incision was made, and the sheath was inserted through the incision. The resistor feature was then attached to the sheath magnetically as described above. Tissue glue (3M Vetbond, part number 1469SB) was applied so that the resistor would not move with respect to the bowel during testing. Once the resistor was affixed, the sensor housing was inserted into the sheath and pushed forward until the housing magnet was aligned the top magnet of the resistor attachment.

Once the resistor was in place, negative control trials were performed. For the negative control, the sensor fixture was inserted into the sheath, data collection began, and the sensor fixture (housing and sensor array) was run beneath the resistor. At least one negative control trial was run at each location.

Following the negative control trial, the heat spot was established. A magnet was placed in the top hole of the resistor fixture (FIG. 21A). The sensor fixture was then pushed through the sheath until the magnet was aligned with the resistor as indicated by the magnetic forces (FIGS. 21D and E).

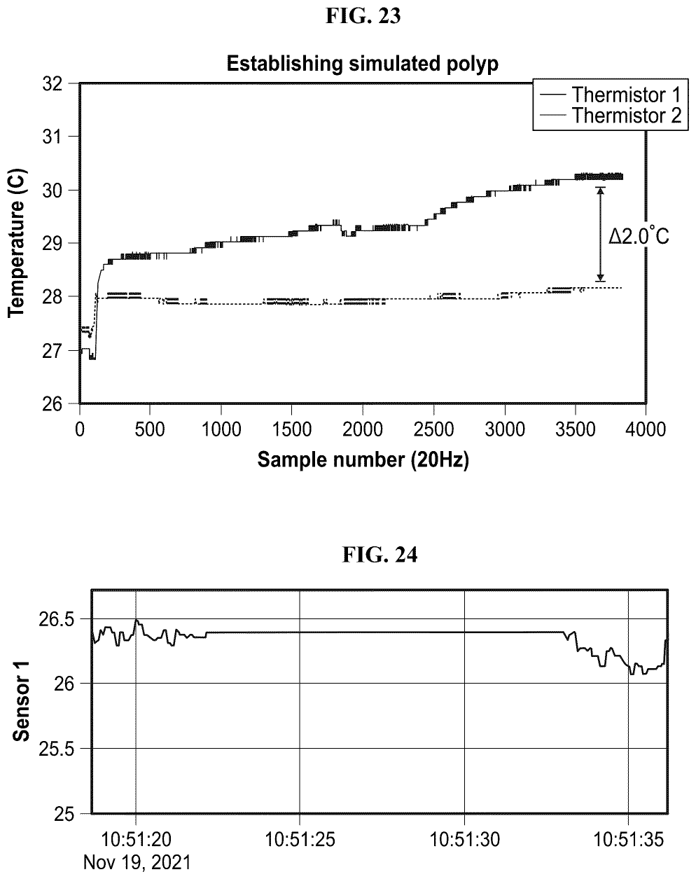

Once the sensor fixture was positioned, a voltage was applied to the resistor in order to begin resistive heating and abnormality simulation. The difference in measurement between Thermistor 1 and Thermistor 2 was monitored and the voltage was adjusted in real time until a steady state difference was obtained. FIG. 23 provides an exemplary measurement of the two thermistors as the voltage is altered to result in a steady state Δ of 2.0° C. Once the desired temperature difference was obtained, the top magnet was removed to allow free movement of the sensor fixture within the sheath.

Between two and ten trial runs were completed at each of the nine locations. Each trial run contained between one and three passes beneath the simulated abnormality. For blind experiments (or blind trial), additional observers were located in a separate room observing livestream data in the GUI, where the observer would mark what they thought was a “polyp” based on rising and falling of FAR-IR data. To assess challenge conditions, 5 mL of saline water was injected onto the inside of the bowel wall at the site of the heat spot immediately before the test run or fecal matter was applied to the sensor face. All measurements were taken with a fixed distance of 0.1″ (0.254 cm) from the mucosal surface.

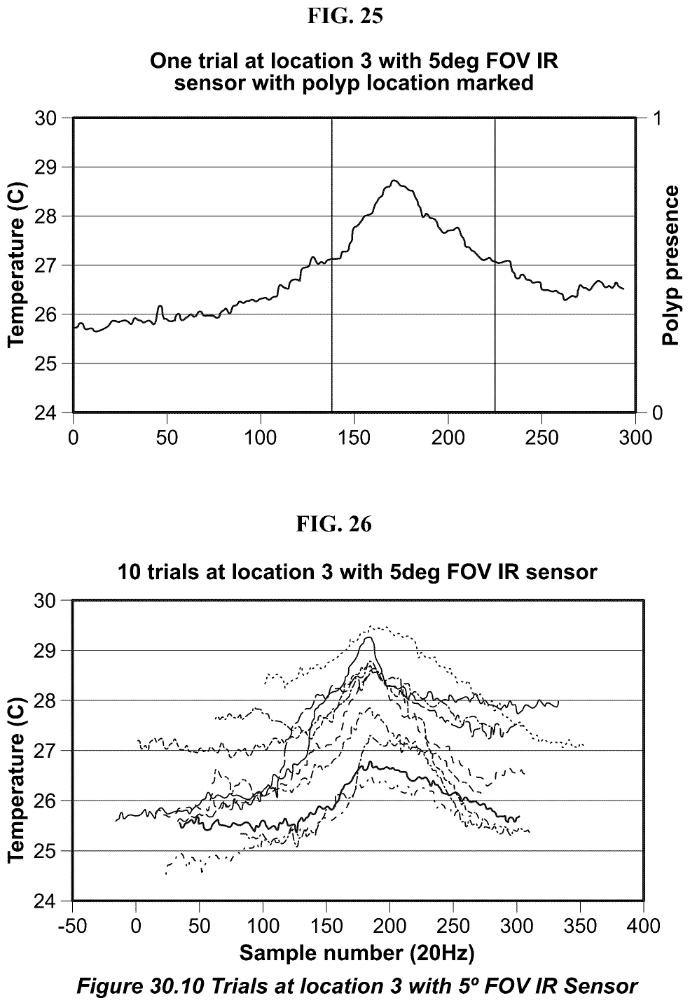

Locations 1-5 were used to test 5° and 12° FOV sensor function. Following the negative control, and establishing a 2° Δ in Temperature, measurements began. FIG. 24 provides an exemplary negative control run. For the 5° FOV measurements, the sensor was passed over the simulated abnormality ten times. FIG. 25 is an exemplary experiment at location 3, where the positions as the abnormality entered the FOV of the sensor are indicated by the vertical lines.

FIG. 26 provides the FAR-IR data for each of the 10 trial runs at location 3. Each trial successfully demonstrated the ability of the FAR-IR sensor to recognize the temperature change associated with the simulated abnormality.

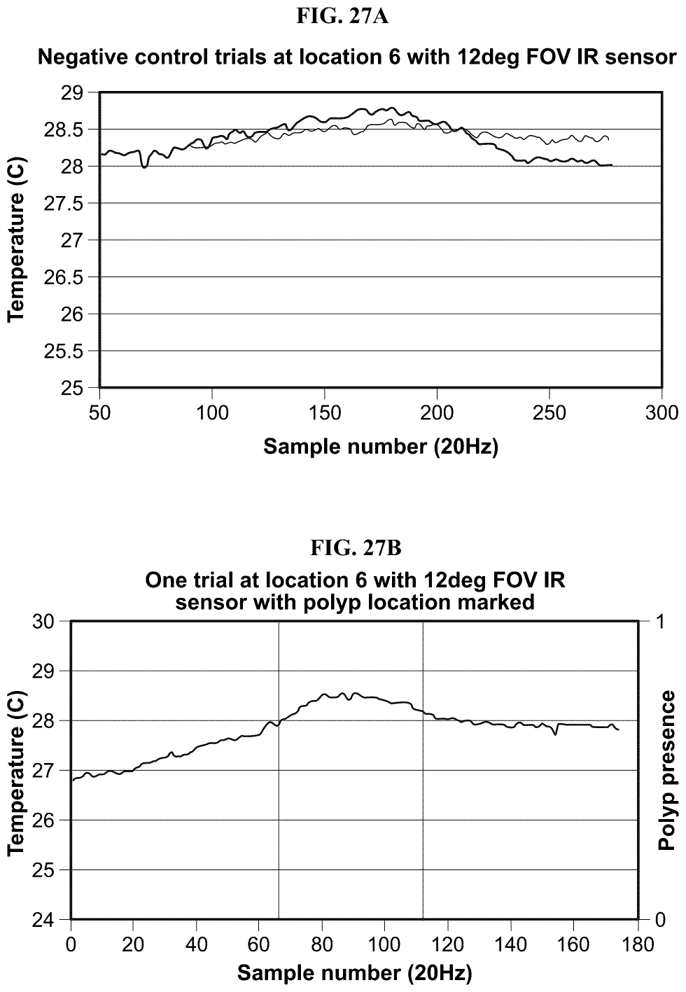

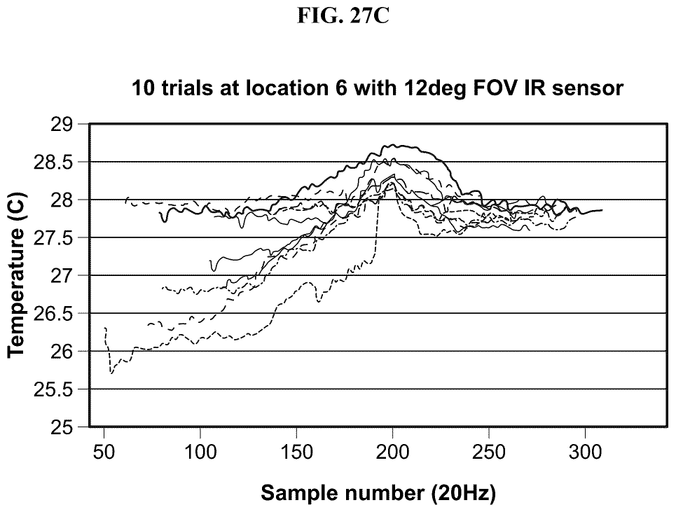

FIGS. 27A-C provide the negative control measurements at location 6 with the 12° FOV sensor (FIG. 27A), an exemplary run at location 6 with the 12° FOV sensor with vertical lines indicating the positions when the abnormality entered and exited the FOV of the sensor (FIG. 27B), and data from the ten runs at location 6 (FIG. 27C). Like the 5° FOV sensor measurements, most measurements illustrate a temperature spike associated with abnormality detection. Although the 12° FOV sensor measurements were less sensitive and showed smaller temperature deviations than the 5° FOV measurements, these sensors were also able to accurately identify abnormality presence within the FOV.

### Controlled Blind Study

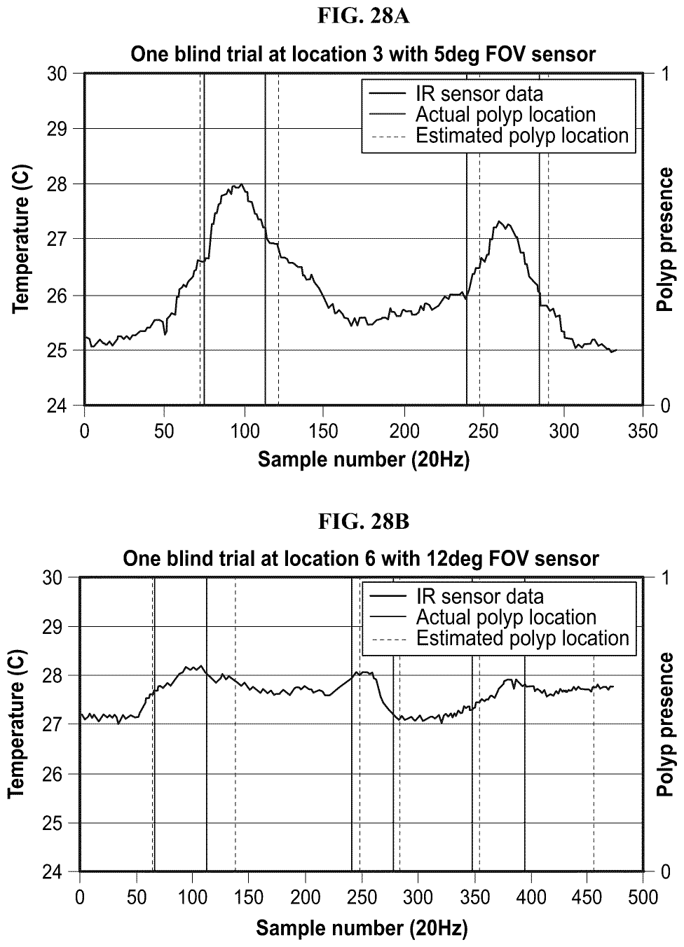

Ten blind trial runs were conducted at three separate locations (2, 3, and 6) using both types of sensors. For almost all trials, the correct number of simulated abnormalities was detected by the unbiased observers marking data in the live data stream to the GUI when the observer believed the abnormality was being detected and when it was not (i.e., the estimated location). The observers were unaware of when the abnormality would appear in the trial data nor how many passes over the abnormality would be made. Each of the trials contained between one and three abnormality passes. FIGS. 28A and B provide data from individual trials correlating the unbiased observation of abnormality detection (dashed vertical lines) with the actual location (solid vertical lines). As can be seen, the observers were able to accurately identify the presence of subsurface heat anomalies using the FAR-IR data.

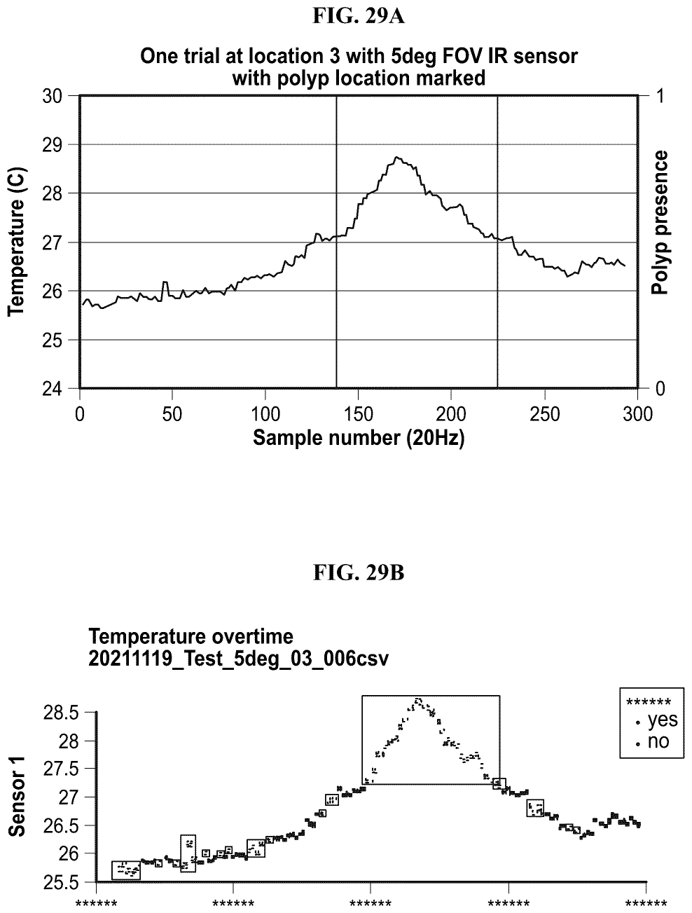

This kind of information may be sent and used in the machine training algorithms described herein. FIG. 29A provides a measurement at location 3 with a 5° FOV FAR-IR sensor. A machine learning algorithm was developed based on the data collected herein and applied to the raw data provided in 29A in order to positions of abnormality presence (or “outliers”). FIG. 29B provides the results of this data being analyzed by the machine learning algorithm where the boxed data illustrates the data identified by the calculation unit as being an outlier. In addition to certain noise fluctuations, the algorithm was able to correctly identify the abnormality position. It is expected that larger data sets, in addition to optimization of device geometry, and increased machine learning will decrease the outlier detection rate.

A double-blind control study was also performed to attempt to identify heat sources in which neither the passive observer nor the GUI operator had knowledge of when the sensor encountered the simulated abnormality. Data from these measurements illustrate that the sensor detected changes temperature from the simulated abnormality, despite inability to always precisely control temperature differences and the unknown location of the abnormality. For these measurements, the bowel was placed back inside the abdominal cavity, resulting in a higher overall temperature than previously described measurements and a 3° Δ in temperature.

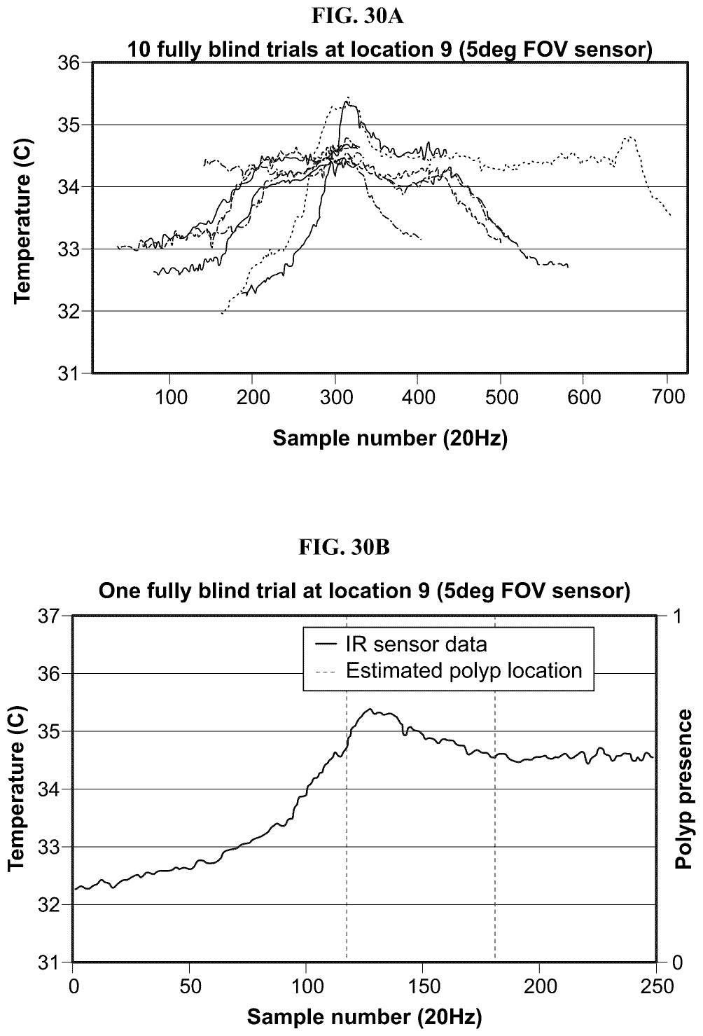

FIG. 30A shows the results of 10 fully blind trial runs at location 9 using a 5° FOV sensor. Significant temperature variation is observed which has similar structure to the measurements performed when abnormality detection could be observed. Variability in these measurements may also come from decreased bowel integrity due to multiple use of the tissue and thickening of the bowel wall and minimal track length which inhibited the identification of a baseline temperature in some measurements. Nevertheless, the peak of the A in temperature as measured from the FAR-IR sensor was measured at 3° C., a temperature difference associated with the resistor attached to the mucosal surface. FIG. 30B provides one of the test runs from where a GUI operator estimated the abnormality location based on the fully blind data collection.

### Stressor Tests

After affixing the resistor to a location exterior to the colon, at either location 3 or 6, running the negative control, and setting the heat spot temperature to two degrees above baseline tissue temperature as determined from Thermistor 2, 5 mL of saline was applied to the internal bowel surface directly beneath the simulated abnormality. Following application, the sensor was passed across the simulated abnormality as described above. Stressor measurements were performed on both 5° FOV and 12° FOV sensors.

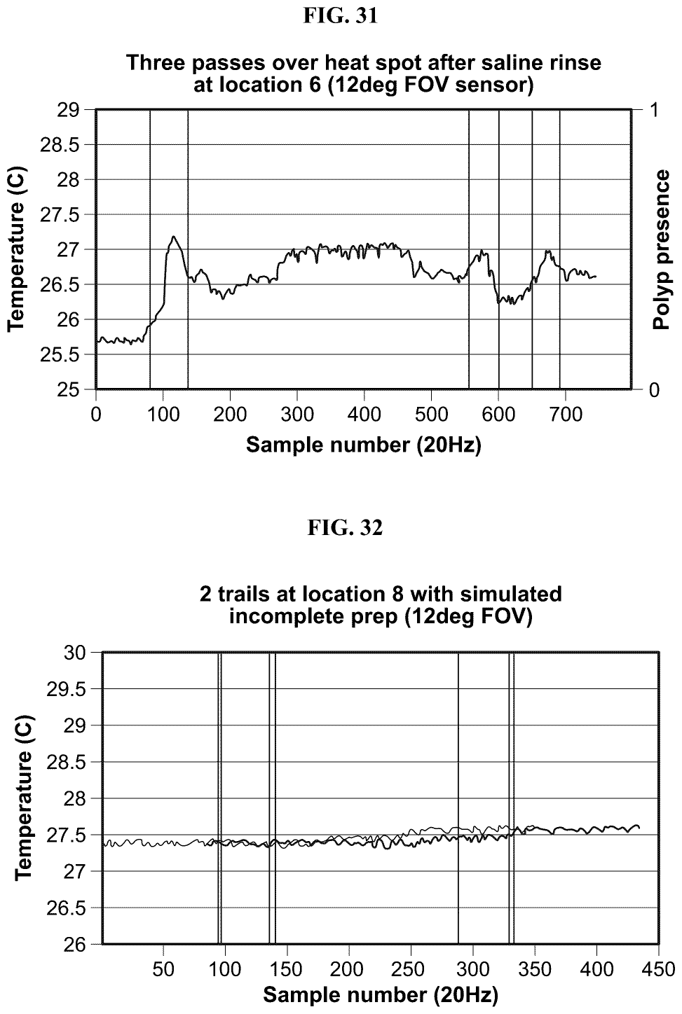

FIG. 31 provides a measurement involving sensor passing over the simulated abnormality following the saline wash. Although sensitivity in these measurements was decreased, the simulated abnormality could be clearly identified from the FAR-IR data.

Additional stressor tests were performed by applying fecal matter to the face of the FAR-IR sensor at location 8. FIG. 32 provides the data of two trials at location 8. As can be seen, the presence of fecal matter on the sensor face, a likely variable encountered during colonoscopies, abrogates the simulated abnormality signal.

### Example 5: Polyp Temperature Profile Detection

The temperature of polyps in the GI tract were evaluated during a routine colonoscopy and was compared with surrounding normal tissues similar to the analysis of increased temperature anomaly found Stefanadis, C. Journal of Clinical Gastroentereology 36.3 (2003):215-218 and Banic, M. Periodicum biologorum 113.4 (2011):439-444, each of which are hereby incorporated by reference in their entirety. The temperature in the center of an identified polyp was performed following measurement of temperature in the surrounding normal tissue 1-2 inches from the polyp.

Temperature measurements were performed using model HSTH-44000 Series Thermistors obtained from Omega. One thermistor per patient was utilized. A data logger, from Omega, was connected to the thermistor and a laptop for image generation and reading and analysis of the temperature data. The colonoscope was provided by Charlottesville Gastroenterology Associates.

Routine colonoscopies were performed with the colonoscope comprising a thermistor for contact thermal measurement of tissue in the GI tract. Patients were excluded if they had cancer identified during the colonoscopy or they had a known history of inflammatory bowel disease. The thermistor was placed through the working channel of the colonoscope. Following identification of one or more polyps using the colonoscopy image, the clean thermisistor was contacted with the polyp for 15-30 seconds. Additionally, the thermistor was brought into the surrounding tissues 2-3 times for 15-30 seconds each time of contact.

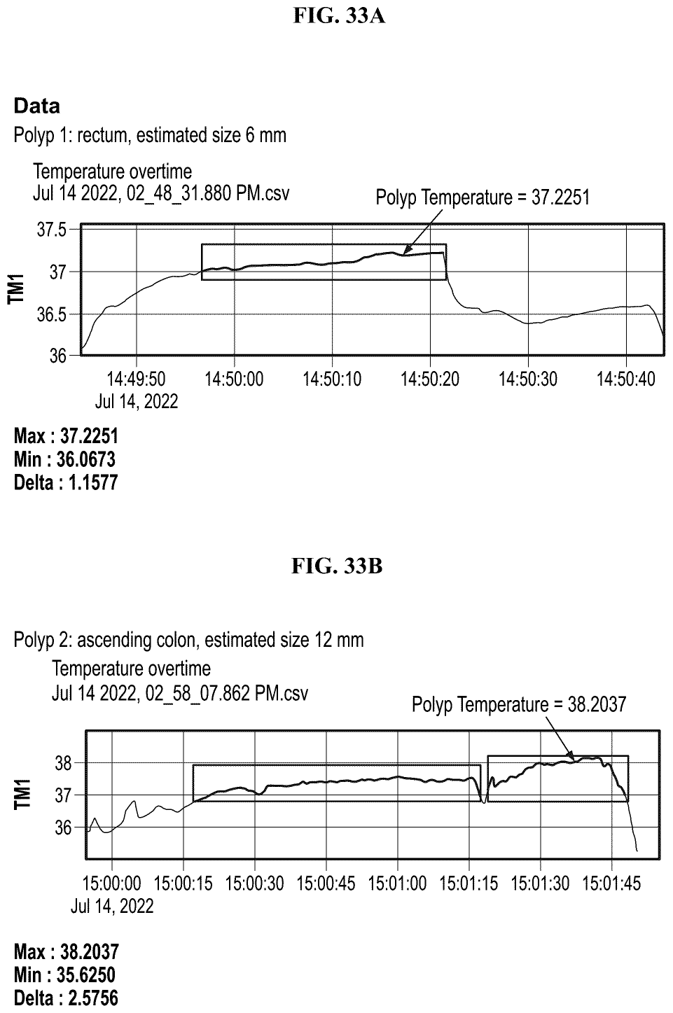

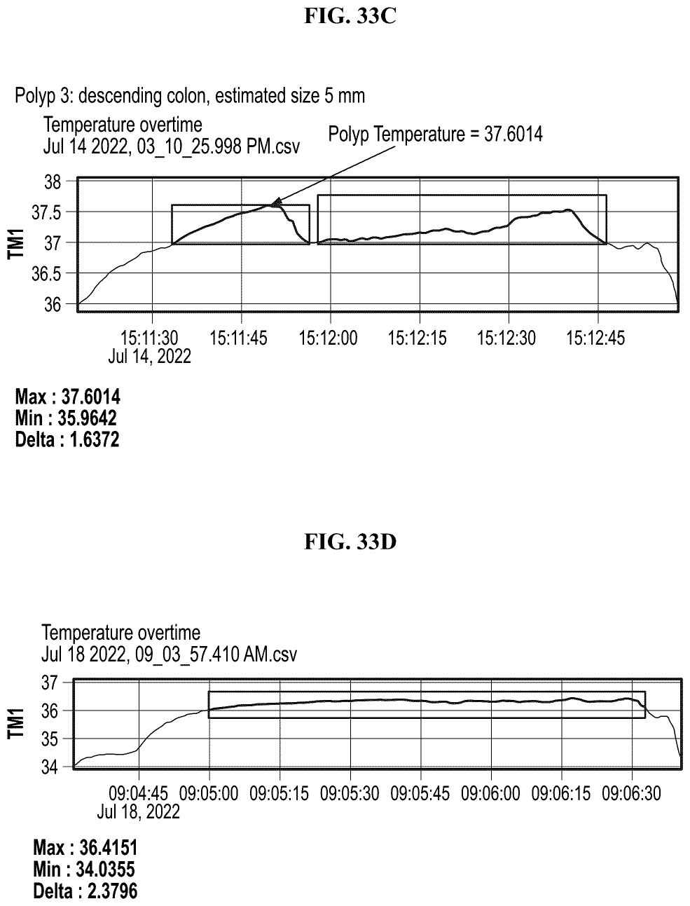

These augmented colonoscopies were performed on seven patients over two days. One patient had 3 polyps identified during the colonoscopy. The first polyp was identified in the rectum was 6 mm in diameter measuring 37.2° C. with comparative surrounding tissue of 36.0° C. FIG. 33A provides the temperature measurements surrounding the polyp as measured in Celsius by the thermistor (TM1). The second polyp was identified in the ascending colon was 12 mm in diameter. At the base of the stalk, the temperature was 38° C. while on the top portion of the pedunculated polyp, the temperature was 37.5° C. The surrounding tissue was 36.0° C. to 37.2° C. and measurements may have been compromised by the presence of water on the dependent tissue. FIG. 33B provides the temperature measurements surrounding the polyp as measured in Celsius by the thermistor (TM1). The third polyp was located in the descending colon and had a 5 mm diameter with a temperature of 37.5° C. as compared to normal surrounding tissue having a temperature of 36.0° C. FIG. 33C provides the temperature measurements surrounding the polyp as measured in Celsius by the thermistor (TM1). Another patient had a single polyp in the transverse colon with an estimated diameter of 3 mm with a temperature of 36.5° C. as compared to 36.0° C. in the surrounding tissue. FIG. 33D provides the temperature measurements surrounding the polyp as measured in Celsius by the thermistor (TM1).

Without wishing to be bound by theory, polyps located in any part of the colon (e.g., ascending, transverse, descending, rectosigmoid) have a higher temperature than the surrounding tissue in the respective location of each polyp. An average of 1.215° C. difference between an anomaly (e.g., polyp, subcutaneous abnormality) and normal mucosal tissue was identified providing confirmatory evidence of this hypothesis.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein, and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. A tip section of the tubular portion of an endoscope, wherein the tip section comprises the distal end of the endoscope tube and the wall of the tube proximal thereto; said tip section comprising:
a) a plurality of far-infrared sensors and/or one or more temperature sensors independently distributed around and/or within said wall;
b) a camera positioned on the distal end for imaging objects in front of the tube (e.g., the field of view of the camera includes the major longitudinal axis of the tube that extends past the distal end);
wherein the plurality of far-infrared sensors, one or more temperature sensors, and camera are configured each to transmit data for processing and analysis in conjunction with one another.

2. The tip section according to claim 1, wherein the field of view of at least one infrared sensor includes an axis perpendicular to the wall surface or perpendicular to the longitudinal axis of the tube.

3. The tip section according to any one of claim 1 or 2, wherein the field of view of each of the plurality of far-infrared sensors and the camera do not overlap.

4. The tip section according to any one of claim 1 or 2, wherein the field of view of at least one of the sensors of plurality of far-infrared sensors and the camera overlap.

5. The tip section according to any one of claims 1-4, wherein the field of view of at least two of said plurality of far-infrared sensors overlap.

6. The tip section according to any one of claims 1-5, wherein at least one of the far-infrared sensors has a field of view less than (or from 0.1° to) 25° (e.g., less than 23°, less than 18°, less than 15°, from 1° to 20°, from 4° to 13°, from 5° to 12°).

7. The tip section according to any one of claims 1-6, wherein at least 50% (e.g., at least 60%, at least 70%, at least 80%, at least 90%, all) of said plurality of far-infrared sensors independently have a field of view less than (or from 0.1° to) 25° (e.g., less than 22°, less than 18°, less than 15°, from 3° to 22°, from 1° to 20°, from 4° to 13°, from 5° to 12°, from 1° to 2°, from 2° to 3°, from 3° to 4°, from 4° to 5°, from 5° to 6°, from 6° to 7°, from 7° to 8°, from 8° to 9°, from 9° to 10°, from 10° to 11°, from 11° to 12°, from 12° to 13°, from 13° to 14°, from 14° to 15°, from 15° to 16°, from 16° to 17°, from 17° to 18°, from 18° to 19°, from 19° to 20°, from 20° to 21°, from 21° to 22°, from 22° to 23°, from 23° to 24°).

8. The tip section according to any one of claims 1-7, wherein at least 90% (e.g., all) of said plurality of far-infrared sensors independently have a field of view from 1° to 20° (e.g., 5° to 12°).

9. The tip section according to any one of claims 1-8, wherein a portion (e.g., a first portion, a second portion) of said plurality of sensors are disposed around or partially around the circumference of the wall.

10. The tip section according to any one of claims 1-9, wherein a portion (e.g., a first portion, a second portion) of said plurality of sensors are embedded in the circumference of the wall.

11. The tip section according to claim 10, wherein said circumference is perpendicular to the major longitudinal axis of the tube.

12. The tip section according to any one of claims 1-11, wherein a portion (e.g., a first portion, a second portion) of said plurality of sensors are disposed linearly along the wall such that said linear dispersion is substantially parallel (e.g., ±5°, ±1°) with the horizontal axis of the tube.

13. The tip section according to any one of claims 1-12, wherein the tube is a cylindrical tube and/or a tube having straight and/or curved edges.

14. The tip section according to claim 13, wherein said cylindrical tube is an elliptic cylinder.

15. The tip section according to claim 13, wherein said cylindrical tube is a circular cylinder.

16. A device for attachment to a tubular portion of an endoscope, wherein the device for tip section augmentation is attachable (e.g., removably attached) to a wall proximal to the distal end of an endoscope tube, and the endoscope comprises a camera positioned on the distal end for imaging objects in front of the tube (e.g., the field of view of the camera includes the major longitudinal axis of the tube that extends past the distal end);
said device for tip section augmentation comprises a plurality of far-infrared sensors and/or one or more temperature sensors distributed on a substrate; and said substrate is attachable to the wall proximal to the distal end of the endoscope; and
wherein the plurality of far-infrared sensors, one or more temperature sensors, and camera are configured each to transmit data for processing and analysis in conjunction with one another.

17. The device according to claim 16, wherein the field of view of at least one infrared sensor includes an axis perpendicular to the wall surface or perpendicular to the longitudinal axis of the tube.

18. The device according to any one of claim 16 or 17, wherein the field of view of each of the plurality of far-infrared sensors and the camera do not overlap when said device is attached to the endoscope.

19. The device according to any one of claim 16 or 17, wherein the field of view of at least one of the plurality of far-infrared sensors and the camera overlap when said device is attached to the endoscope.

20. The device according to any one of claims 16-20, wherein the field of view of at least two of said plurality of far-infrared sensors overlap.

21. The device according to any one of claims 16-21, wherein at least one of the far-infrared sensors has a field of view less than (or from 0.1° to) 20° (e.g., less than 18°, less than 15°, from 1° to 20°, from 4° to 13°, from 5° to 12°).

22. The device according to any one of claims 16-21, wherein at least 50% (e.g., at least 60%, at least 70%, at least 80%, at least 90%, all) of said plurality of far-infrared sensors independently have a field of view less than (or from 0.1° to) 25° (e.g., less than 22°, less than 18°, less than 15°, from 3° to 22°, from 1° to 20°, from 4° to 13°, from 5° to 12°, from 1° to 2°, from 2° to 3°, from 3° to 4°, from 4° to 5°, from 5° to 6°, from 6° to 7°, from 7° to 8°, from 8° to 9°, from 9° to 10°, from 10° to 11°, from 11° to 12°, from 12° to 13°, from 13° to 14°, from 14° to 15°, from 15° to 16°, from 16° to 17°, from 17° to 18°, from 18° to 19°, from 19° to 20°, from 20° to 21°, from 21° to 22°, from 22° to 23°, from 23° to 24°).

23. The device according to any one of claims 16-22, wherein at least 90% (e.g., all) of said plurality of far-infrared sensors independently have a field of view from 1° to 20° (e.g., 5° to 12°).

24. The device according to any one of claims 16-23, wherein a portion (e.g., a first portion, a second portion) of said plurality of sensors are disposed around or partially around the circumference of the wall when said device is attached to the endoscope.

25. The device according to claim 24, wherein said circumference is perpendicular to the major longitudinal axis of the tube when said device is attached to the endoscope.

26. The device according to any one of claims 16-25, wherein a portion (e.g., a first portion, a second portion) of said plurality of sensors are disposed linearly along the wall such that said linear dispersion is substantially parallel (e.g., ±5°, ±1°) with the horizontal axis of the tube when said device is attached to the endoscope.

27. The device according to any one of claims 16-26, wherein the tube is a cylindrical tube and the substrate is dimensioned for attachment thereto.

28. The device according to claim 27, wherein said cylindrical tube is an elliptic cylinder (e.g., the substrate is elliptic).

29. The device according to claim 27, wherein said cylindrical tube is a circular cylinder (e.g., the substrate is circular).

30. An endoscope system comprising:
a) an endoscope having the tip section according to any one of claims 1-15 or the device according to any one of claims 16-29 attached thereto;
b) a machine readable medium configured to receive the data transmitted from the plurality of far-infrared sensors and, optionally, the camera; and
c) a processor comprising instructions to analyze the data transmitted to the machine readable medium to identify abnormalities of an organ or series of organs (e.g., the GI tract of a subject) based on the far-IR sensor data and, optionally, the image provided (e.g., conditions that exist on the tissue surface of the organ such as polyps, conditions that exist below the tissue surface, tumors, cysts, granulomas, abnormal circulatory vessels, inflammation).

31. The endoscope system according to claim 30, wherein the processor comprises instructions for a camera ML/AI algorithm to detect abnormalities from the camera images transmitted to the machine readable medium.

32. The endoscope system according to claim 30 or 31, wherein the processor comprises instructions for a far-infrared AI algorithm to detect abnormalities data transmitted from the plurality of sensors to the machine readable medium.

33. The endoscope system according to claim 32, wherein the processor comprises instructions for comparing the output of the camera ML/AI algorithm and the far-infrared ML/AI algorithm.

34. The endoscope system according to any one of claims 30-33, wherein the instructions to analyze the data and identify abnormalities includes a calculation involving the position of the distal tip in the GI tract and/or movement speed of the distal tip during data collection.

35. The endoscope system according to any one of claims 30-34, wherein the processor comprises instructions to analyze data transmitted from the plurality of far-infrared sensors to identify subcutaneous abnormalities.

36. A method using an endoscope system to view an object comprising:
a) positioning an endoscope having a plurality of far-infrared sensors and/or temperature sensors distributed around a wall proximal to the distal end of the tube; wherein the endoscope is positioned such that the object is in the field of view of at least one of the plurality of far-infrared sensors and/or temperature sensors to detect heat and/or temperature data of the object; and
b) transmitting the heat and/or temperature data to a machine readable medium.

37. A method using an endoscope system to view an object comprising:
a) providing an endoscope having the tip section according to any one of claims 1-15 or the device according to any one of claims 16-29 attached thereto;
b) positioning a light source capable of emitting light (e.g., white light, red light, blue light, green light, infrared light, near-infrared light), wherein light emitted from the light source is reflected off the object and into the camera to form image data;
c) transmitting the image data to a machine readable medium;
d) optionally moving the distal tip of the endoscope such that the object is in the field of view of one or more of the far-infrared sensors to detect heat data of the object; and
e) transmitting the heat and/or temperature data from the plurality of far-IR sensors and/or temperature sensors to a machine readable medium.

38. A method using an endoscope system to view an object comprising:
a) positioning an endoscope having the tip section according to any one of claims 1-15 or the device according to any one of claims 16-29 attached thereto, wherein the endoscope is positioned such that the object is in the field of view of at least one of the plurality of far-infrared sensors to detect heat and/or temperature data of the object;
b) transmitting the heat and/or temperature data to a machine readable medium;
c) positioning a light source capable of emitting light (e.g., white light, red light, blue light, green light, infrared light, near-infrared light), wherein light emitted from the light source is reflected off the object and into the camera to form image data;
d) transmitting the image data to a machine readable medium.

39. The method according to claim 37 or 38, wherein the light source is positioned by movement of the distal tip.

40. The method according to any one of claims 36-39, wherein the distal tip is moved such that the object passes through the field of view of at least two far-infrared sensors in the plurality of far-infrared sensors.

41. The method according to claim 40, wherein heat data from each sensor is collected during said movement and transmitted to said machine readable medium.

42. The method according to any one of claims 37-41, wherein said machine readable medium is in communication with a processor comprising instructions to analyze the heat data and/or image data.

43. The method according to any one of claims 37-42, wherein the heat and/or temperature data identifies an abnormality (e.g., polyp, subcutaneous abnormality) in a location (e.g., location of tissue) and the light source is positioned to reflect off the abnormality identified and into the camera.

44. The method according to claim 43, wherein the method further comprises rinsing the location.

45. The method according to claim 44, wherein the rinsing occurs before positioning the light source to view the abnormality location.

46. The method according to claim 44, wherein the rinsing occurs after positioning the light source to view the abnormality location, and the method further comprises re-positioning the light source to reflect off the abnormality identified and into the camera to view the rinsed abnormality location.

47. The method according to any one of claims 37-46, further comprising transmitting and/or displaying the heat and/or temperature and/or image data to an interface (e.g., a graphical user interface) for an endoscope user to view (e.g., in real-time) an object identified in the data (e.g., an abnormality, a rinsed abnormality, a potential abnormality).
